(19) United States
(12) Patent Application Publication
(10) Pub. No.: US 2017/0095192 A1
(43) Pub. Date: Apr. 6, 2017

(54) MENTAL STATE ANALYSIS USING WEB SERVERS

(71) Applicant: Affectiva, Inc., Waltham, MA (US)

(72) Inventors: Richard Scott Sadowsky, Sturbridge, MA (US); Rana el Kaliouby, Milton, MA (US); Rosalind Wright Picard, Newtonville, MA (US); Oliver Orion Wilder-Smith, Holliston, MA (US); Panu James Turcot, Pacifica, CA (US); Zhihong Zeng, Lexington, MA (US)

(73) Assignee: Affectiva, Inc., Waltham, MA (US)

(21) Appl. No.: 15/382,087

(22) Filed: Dec. 16, 2016

Related U.S. Application Data
(63) Continuation-in-part of application No. 13/153,745, filed on Jun. 6, 2011.
Application No. 13/153,745 is a continuation-in-part of application No. 15/262,197, filed on Sep. 12, 2016.
Application No. 15/262,197 is a continuation-in-part of application No. 14/796,419, filed on Jul. 10, 2015.
Application No. 14/796,419 is a continuation-in-part of application No. 13/153,745, filed on Jun. 6, 2011.
Application No. 14/796,419 is a continuation-in-part of application No. 14/460,915, filed on Aug. 15, 2014.
Application No. 14/460,915 is a continuation-in-part of application No. 13/153,745, filed on Jun. 6, 2011.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 62/217,872, filed on Sep. 12, 2015.
(60) Provisional application No. 62/222,518, filed on Sep. 23, 2015.
(60) Provisional application No. 62/265,937, filed on Dec. 10, 2015.
(60) Provisional application No. 62/273,896, filed on Dec. 31, 2015.
(60) Provisional application No. 62/301,558, filed on Feb. 29, 2016.
(60) Provisional application No. 62/370,421, filed on Aug. 3, 2016.
(60) Provisional application No. 62/023,800, filed on Jul. 11, 2014.
(60) Provisional application No. 62/047,508, filed on Sep. 8, 2014.
(60) Provisional application No. 62/082,579, filed on Nov. 20, 2014.
(60) Provisional application No. 62/128,974, filed on Mar. 5, 2015.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/867,007, filed on Aug. 16, 2013.
(60) Provisional application No. 61/924,252, filed on Jan. 7, 2014.
(60) Provisional application No. 61/916,190, filed on Dec. 14, 2013.
(60) Provisional application No. 61/927,481, filed on Jan. 15, 2014.
(60) Provisional application No. 61/953,878, filed on Mar. 16, 2014.
(60) Provisional application No. 61/972,314, filed on Mar. 30, 2014.
(60) Provisional application No. 62/023,800, filed on Jul. 11, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/16; A61B 5/0205; A61B 5/00

(52) U.S. Cl. CPC A61B 5/165; A61B 5/0013; A61B 5/0022; A61B 5/0077; A61B 5/0205; A61B 2562/0219; A61B 2576/00; A61B 5/02405

(57) ABSTRACT

Analysis of mental states is provided using web servers to enable data analysis. Data is captured for an individual where the data includes facial information and physiological information. Data that was captured for the individual is compared against a plurality of mental state event temporal signatures. Analysis is performed on a web service and the analysis is received. The mental states of other people are correlated to the mental state for the individual. Other sources of information are aggregated, where the information is used to analyze the mental state of the individual. Analysis of the mental state of the individual or group of individuals is rendered for display.

### RELATED APPLICATIONS

This application is a continuation in part of “Mental State Analysis using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

This application is also a continuation-in-part of U.S. patent application “Mental State Event Signature Usage” Ser. No. 15/262,197, filed Sep. 12, 2016, which claims the benefit of U.S. provisional patent applications “Mental State Event Signature Usage” Ser. No. 62/217,872, filed Sep. 12, 2015, “Image Analysis In Support of Robotic Manipulation” Ser. No. 62/222,518, filed Sep. 23, 2015, “Analysis of Image Content with Associated Manipulation of Expression Presentation” Ser. No. 62/265,937, filed Dec. 10, 2015, “Image Analysis Using Sub-Sectional Component Evaluation To Augment Classifier Usage” Ser. No. 62/273,896, filed Dec. 31, 2015, “Analytics for Live Streaming Based on Image Analysis within a Shared Digital Environment” Ser. No. 62/301,558, filed Feb. 29, 2016, and “Deep Convolutional Neural Network Analysis of Images for Mental States” Ser. No. 62/370,421, filed Aug. 3, 2016. The patent application “Mental State Event Signature Usage” Ser. No. 15/262,197, filed Sep. 12, 2016, is also a continuation-in-part of U.S. patent application “Mental State Event Definition Generation” Ser. No. 14/796,419, filed Jul. 10, 2015, which claims the benefit of U.S. provisional patent applications “Mental State Event Definition Generation” Ser. No. 62/023,800, filed Jul. 11, 2014, “Facial Tracking with Classifiers” Ser. No. 62/047,508, filed Sep. 8, 2014, “Semiconductor Based Mental State Analysis” Ser. No. 62/082,579, filed Nov. 20, 2014, and “Viewership Analysis Based On Facial Evaluation” Ser. No. 62/128,974, filed Mar. 5, 2015. The patent application “Mental State Event Definition Generation” Ser. No. 14/796,419, filed Jul. 10, 2015 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The patent application “Mental State Event Definition Generation” Ser. No. 14/796,419, filed Jul. 10, 2015 is also a continuation-in-part of U.S. patent application “Mental State Analysis Using an Application Programming Interface” Ser. No. 14/460,915, Aug. 15, 2014, which claims the benefit of U.S. provisional patent applications “Application Programming Interface for Mental State Analysis” Ser. No. 61/867,007, filed Aug. 16, 2013, “Mental State Analysis Using an Application Programming Interface” Ser. No. 61/924,252, filed Jan. 7, 2014, “Heart Rate Variability Evaluation for Mental State Analysis” Ser. No. 61/916,190, filed Dec. 14, 2013, “Mental State Analysis for Norm Generation” Ser. No. 61/927,481, filed Jan. 15, 2014, “Expression Analysis in Response to Mental State Express Request” Ser. No. 61/953,878, filed Mar. 16, 2014, “Background Analysis of Mental State Expressions” Ser. No. 61/972,314, filed Mar. 30, 2014, and “Mental State Event Definition Generation” Ser. No. 62/023,800, filed Jul. 11, 2014. The patent application “Mental State Analysis Using an Application Programming Interface” Ser. No. 14/460,915, Aug. 15, 2014 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. Each of the foregoing applications is hereby incorporated by reference in its entirety.

### FIELD OF INVENTION

This application relates generally to analysis of mental states and more particularly to evaluation of mental states using image analysis and signatures using web servers.

### BACKGROUND

The evaluation of mental states is key to understanding individuals but is also useful for therapeutic and business purposes. Mental states run a broad gamut from happiness to sadness, from contentedness to worry, and from excited to calm, among many others. These mental states are experienced in response to everyday events such as frustration during a traffic jam, boredom while standing in line, and impatience while waiting for a cup of coffee. Individuals can perceive and empathize with other people by evaluating and understanding their mental states, but automated evaluation of mental states is far more challenging. An empathetic person might perceive someone else feeling anxious or joyful and respond accordingly. The ability and means by which one person perceives another's emotional state is often quite difficult to summarize and has sometimes been described as having a “gut feeling”.

Many mental states, such as confusion, concentration, and worry, can be identified to aid in the understanding of an individual or a group of people. For example, in the aftermath of a catastrophe, people can collectively respond with fear or anxiety. Likewise, people can collectively respond with happy enthusiasm when their sports team obtains a victory, for instance. Certain facial expressions and head gestures can be used to identify a mental state that a person is experiencing. Limited automation has been performed in the evaluation of mental states based on facial expressions. Certain physiological conditions provide telling indications of a person's state of mind and have been used in a crude fashion, such as for lie detector or polygraph tests.

Gaining insight into the mental states of multiple individuals represents an important tool for understanding events. For example, advertisers seek to understand the resultant mental states of viewers of their advertisements in order to gauge the efficacy of those advertisements. However, it is very difficult to properly interpret mental states when the individuals under consideration might themselves be unable to accurately communicate their mental states. Adding to the difficulty is the fact that multiple individuals can have similar or very different mental states when taking part in the same shared activity. For example, the mental state of two friends can be very different after a certain team wins an important sporting event. Clearly, if one friend is a fan of the winning team, and the other friend is a fan of the losing team, widely varying mental states can be expected. However, the problem of defining the mental states of more than one individual to stimuli more complex than a sports team winning or losing can prove a much more difficult exercise in understanding mental states. Thus there remains a need for improved evaluation of mental states in an automated fashion.

### SUMMARY

A computer implemented method for analyzing mental states is disclosed comprising: capturing data on an individual into a computer system, wherein the data provides information for evaluating a mental state of the individual, and wherein the data includes facial data for the individual; performing image analysis on the facial data, wherein the image analysis includes inferring mental states; comparing the data that was captured for the individual against a plurality of mental state event temporal signatures; receiving analysis from a web server, wherein the analysis is based on the data on the individual which was captured and the comparing the data that was captured for the individual against a plurality of mental state event temporal signatures; and rendering an output which describes the mental state of the individual based on the analysis which was received. The method can further comprise matching a first event signature, from the plurality of mental state event temporal signatures, against the data that was captured. The method can further comprise performing unsupervised clustering of features extracted from the facial data. The method can further comprise analyzing the data to produce mental state information. The analyzing the data can be further based on a demographic basis. The data on the individual can include facial expressions, physiological information, or accelerometer readings. The facial expressions can further comprise head gestures. The physiological information can include electrodermal activity, heart rate, heart rate variability, or respiration. The physiological information can be collected without contacting the individual.

The mental state can be one of a cognitive state and an emotional state. The facial data can include information on facial expressions, action units, head gestures, smiles, squints, lowered eyebrows, raised eyebrows, smirks, and attention. The method can further comprise inferring mental states, based on the data which was collected and the analysis of the facial data. The web server can comprise an interface which includes a cloud-based server that is remote to the individual and cloud-based storage. The web server can comprise an interface which includes a datacenter-based server that is remote to the individual and datacenter-based storage. The method can further comprise indexing the data on the individual through the web server. The indexing can include categorization based on valence and arousal information. The method can further comprise receiving analysis information on a plurality of other people, wherein the analysis information allows evaluation of a collective mental state of the plurality of other people. The analysis information can include correlation for the mental state of the plurality of other people to the data which was captured on the mental state of the individual. The correlation can be based on metadata from the individual and metadata from the plurality of other people. The correlation can be based on the comparing the data that was captured for the individual against a plurality of mental state event temporal signatures.

The analysis which is received from the web server can be based on specific access rights. The method can further comprise sending a request to the web server for the analysis. The analysis can be generated just in time based on the request for the analysis. The method can further comprise sending a subset of the data which was captured on the individual to the web server. The rendering can be based on data which is received from the web server. The data which is received can include a serialized object in a form of JavaScript Object Notation (JSON). The method can further comprise de-serializing the serialized object into a form for a JavaScript object. The rendering can further comprise recommending a course of action based on the mental state of the individual. The recommending can include modifying a question queried to a focus group, changing an advertisement on a web page, editing a movie which was viewed to remove an objectionable section, changing direction of an electronic game, changing a medical consultation presentation, or editing a confusing section of an internet-based tutorial.

In some embodiments, a computer program product stored on a non-transitory computer-readable medium for analyzing mental states, the computer program product comprising code which causes one or more processors to perform operations of: capturing data on an individual into a computer system, wherein the data provides information for evaluating a mental state of the individual, and wherein the data includes facial data for the individual; performing image analysis on the facial data, wherein the image analysis includes inferring mental states; comparing the data that was captured for the individual against a plurality of mental state event temporal signatures; receiving analysis from a web server, wherein the analysis is based on the data on the individual which was captured and the comparing the data that was captured for the individual against a plurality of mental state event temporal signatures; and rendering an output which describes the mental state of the individual based on the analysis which was received. In embodiments, a system for analyzing mental states comprises: a memory which stores instructions; one or more processors attached to the memory wherein the one or more processors, when executing the instructions which are stored, are configured to: capture data on an individual into a computer system, wherein the data provides information for evaluating a mental state of the individual, and wherein the data includes facial data for the individual; perform image analysis on the facial data, wherein the image analysis includes inferring mental states; compare the data that was captured for the individual against a plurality of mental state event temporal signatures; receive analysis from a web server, wherein the analysis is based on the data on the individual which was captured and the comparing the data that was captured for the individual against a plurality of mental state event temporal signatures; and render an output which describes the mental state of the individual based on the analysis which was received.

Various features, aspects, and advantages of various embodiments will become more apparent from the following further description.

### BRIEF DESCRIPTION OF THE DRAWINGS

The following detailed description of certain embodiments can be understood by reference to the following figures wherein:

FIG. 1 is a diagram of a system for analyzing mental states.

FIG. 2 is a flow diagram for obtaining and using data in mental state analysis.

FIG. 3 is a graphical rendering of electrodermal activity.

FIG. 4 is a graphical rendering of accelerometer data.

FIG. 5 is a graphical rendering of skin temperature data.

FIG. 6 shows an image collection system for facial analysis.

FIG. 7 is a flow diagram for performing facial analysis.

FIG. 8 is a diagram describing physiological analysis.

FIG. 9 is a flow diagram describing heart rate analysis.

FIG. 10 is a flow diagram for performing mental state analysis and rendering.

FIG. 11 is a flow diagram describing analysis of the mental response of a group.

FIG. 12 is a flow diagram for identifying data portions which match a selected mental state of interest.

FIG. 13 is a graphical rendering of mental state analysis along with an aggregated result from a group of people.

FIG. 14 is a graphical rendering of mental state analysis.

FIG. 15 is a graphical rendering of mental state analysis based on metadata.

FIG. 16 is a flow diagram for affect-based recommendations.

FIG. 17 shows example image collection including multiple mobile devices.

FIG. 18 illustrates feature extraction for multiple faces.

FIG. 19 shows example facial data collection including landmarks.

FIG. 20 shows example facial data collection including regions.

FIG. 21 is a flow diagram for detecting facial expressions.

FIG. 22 is a flow diagram for large-scale clustering of facial events.

FIG. 23 shows example unsupervised clustering of features and characterizations of cluster profiles.

FIG. 24A shows example tags embedded in a webpage.

FIG. 24B shows example invoking tags for the collection of images.

FIG. 25 shows an example live-streaming social video scenario.

FIG. 26 is a system diagram for analyzing mental state information.

### DETAILED DESCRIPTION

The present disclosure provides a description of various methods and systems for analyzing people's mental states. A mental state can be a cognitive state or an emotional state, and these can be broadly covered using the term affect. Examples of emotional states include happiness or sadness. Examples of cognitive states include concentration or confusion. Observing, capturing, and analyzing these mental states can yield significant information about people's reactions to various stimuli. Some terms commonly used in the evaluation of mental states are arousal and valence. Arousal is an indication on the amount of activation or excitement of a person. Valence is an indication on whether a person is positively or negatively disposed. Determination of affect can include analysis of arousal and valence. Determining affect can also include facial analysis for expressions such as smiles or brow furrowing. Analysis can be as simple as tracking when someone smiles or when someone frowns. Beyond this, recommendations for courses of action can be made based on tracking when someone smiles or demonstrates another affect.

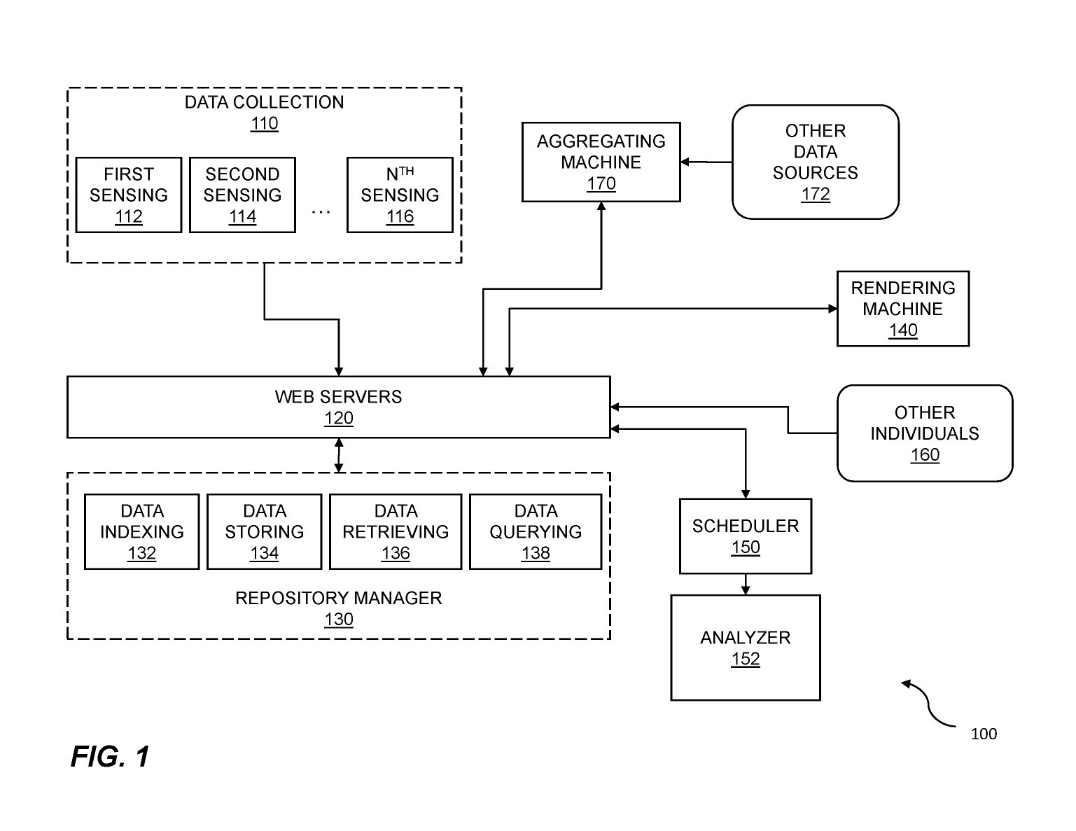

The present disclosure provides a description of various methods and systems associated with performing analysis of mental states. A mental state can be an emotional state or a cognitive state. Examples of emotional states can be happiness or sadness, and examples of cognitive states can be concentration or confusion. FIG. 1 is a diagram of a system 100 for analyzing mental states. The system can include data collection 110, web servers 120, a repository manager 130, an analyzer 152, and a rendering machine 140. The data collection 110 can be accomplished by collecting data from a plurality of sensing structures, such as a first sensing 112, a second sensing 114, through an n^{th} sensing 116. This plurality of sensing structures can be attached to an individual, be near to the individual, or view the individual. These sensing structures can be adapted to perform facial analysis. The sensing structures can be adapted to perform physiological analysis which can include electrodermal activity or skin conductance, accelerometer, skin temperature, heart rate, heart rate variability, respiration, and other types of analysis of a human being. The data collected from these sensing structures can be analyzed in real time or can be collected for later analysis, based on the processing requirements of the needed analysis. The analysis can also be performed “just in time.” A just-in-time analysis can be performed on request, where the result is provided when a button is clicked on in a web page, for instance. Analysis can also be performed as data is collected so that a timeline, with associated analysis, is presented in real time while the data is being collected or with little or no time lag from the collection. In this manner, the analysis results can be presented while data is still being collected on the individual.

The web servers 120 can comprise an interface which includes a server that is remote to the individual and cloud-based storage. Web servers can include a web site, ftp site, or server which provides access to a larger group of analytical tools for mental states. The web servers 120 can also be a conduit for data that was collected as it is routed to other parts of the system 100. The web servers 120 can be a server or a distributed network of computers. The web servers 120 can be cloud-based. The web servers 120 can be datacenter-based. The datacenter-based web server can be remote to the individual and include datacenter-based storage. The web servers 120 can provide a means for a user to log in and request information and analysis. The information request can take the form of analyzing a mental state for an individual in light of various other sources of information or based on a group of people which correlate to the mental state for the individual of interest. In some embodiments, the web servers 120 provide for forwarding data which was collected to one or more processors for further analysis.

The web servers 120 can forward the data which was collected to a repository manager 130. The repository manager can provide for data indexing 132, data storing 134, data retrieving 136, and data querying 138. The data which was collected through the data collection 110, through, for example, a first sensing 112, can be forwarded through the web servers 120 to the repository manager 130. The repository manager can, in turn, store the data which was collected. The data on the individual can be indexed, through web servers, with other data that has been collected on the individual on which the data collection 110 has occurred or can be indexed with other individuals whose data has been stored in the repository manager 130. The indexing can include categorization based on valence and arousal information. The indexing can include ordering based on time stamps or other metadata. The indexing can include correlating the data based on common mental states or based on a common experience of individuals. The common experience can be viewing or interacting with a web site, a movie, a movie trailer, an advertisement, a television show, a streamed video clip, a distance learning program, a video game, a computer game, a personal game machine, a cell phone, an automobile or another vehicle, a product, a web page, consuming a food, and so forth. Other experiences for which mental states can be evaluated include walking through a store, through a shopping mall, or encountering a display within a store.

Multiple ways of indexing can be performed. The data, such as facial expressions or physiological information, can be indexed. One type of index can be a tightly bound index where a clear relationship exists, which might be useful in future analysis. One example is time stamping of the data in hours, minutes, seconds, and perhaps, in certain cases, fractions of a second. Other examples include a project, client, or individual being associated with data. Another type of index can be a looser coupling, where certain possibly useful associations might not be self-evident at the start of an effort. Some examples of these types of indexing include employment history, gender, income, or other metadata. Another example is the location where the data was captured, for instance in the individual's home, workplace, school, or another setting. Yet another example includes information on the person's action or behavior. Instances of this type information include whether a person performed a check-out operation while on a website, whether they filled in certain forms, what queries or searches they performed, and the like. The time of day when the data was captured might prove useful for some types of indexing, as might be the work shift time when the individual normally works. Any sort of information which might be indexed can be collected as metadata. Indices can be formed in an ad hoc manner and retained temporarily while certain analysis is performed. Alternatively, indices can be formed and stored with the data for future reference. Further, metadata can include self-report information from the individuals on which data is collected.

Data can be retrieved through accessing the web servers 120 and requesting data which was collected for an individual. Data can also be retrieved for a collection of individuals, for a given time period, or for a given experience. Data can be queried to find matches for a specific experience, for a given mental response or mental state, or for an individual or group of individuals. Associations can be found through queries and various retrievals which might prove useful in a business or therapeutic environment. Queries can be made based on key word searches, a time frame, or an experience.

In some embodiments, a display is provided using a rendering machine 140. The rendering machine 140 can be part of a computer system which is part of another component of the system 100, part of the web servers 120, or part of a client computer system. The rendering can include graphical display of information collected in the data collection 110. The rendering can include display of video, electrodermal activity, accelerometer readings, skin temperature, heart rate, and heart rate variability. The rendering can also include display of mental states. In some embodiments, the rendering includes probabilities of certain mental states. The mental state for the individual can be inferred based on the data which was collected and can be based on facial analysis of activity units as well as facial expressions and head gestures. For instance, concentration can be identified by a furrowing of eye brows. An elevated heart rate can indicate being excited. Reduced skin conductance can correspond to arousal. These and other factors can be used to identify mental states which might be rendered in a graphical display.

The system 100 can include a scheduler 150. The scheduler 150 can obtain data that came from the data collection 110. The scheduler 150 can interact with an analyzer 152. The scheduler 150 can determine a schedule for analysis by the analyzer 152 where the analyzer 152 is limited by computer processing capabilities where the data cannot be analyzed in real time. In some embodiments, aspects of the data collection 110, the web servers 120, the repository manager 130, or other components of the system 100 require computer processing capabilities for which the analyzer 152 is used. The analyzer 152 can be a single processor, multiple processors, or a networked group of processors. The analyzer 152 can include various other computer components, such as memory and the like, to assist in performing the needed calculations for the system 100. The analyzer 152 can communicate with the other components of the system 100 through the web servers 120. In some embodiments, the analyzer 152 communicates directly with the other components of the system. The analyzer 152 can provide an analysis result for the data which was collected from the individual, wherein the analysis result is related to the mental state of the individual. In some embodiments, the analyzer 152 provides results on a just-in-time basis. The scheduler 150 can request just-in-time analysis by the analyzer 152.

Information from other individuals 160 can be provided to the system 100. The other individuals 160 can have a common experience with the individual on which the data collection 110 was performed. The process can include analyzing information from a plurality of other individuals 160, wherein the information allows evaluation of the mental state of each of the plurality of other individuals 160, and correlating the mental state of each of the plurality of other individuals 160 to the data which was captured and indexed on the mental state of the individual. Metadata can be collected on each of the other individuals 160 or on the data collected on the other individuals 160. Alternatively, the other individuals 160 can have a correlation for mental states with the mental state for the individual on which the data was collected. The analyzer 152 can further provide a second analysis based on a group of other individuals 160, wherein mental states for the other individuals 160 correlate to the mental state of the individual. In other embodiments, a group of other individuals 160 is analyzed with the individual on whom data collection was performed to infer a mental state that is a response of the entire group and is referred to as a collective mental state. This response can be used to evaluate the value of an advertisement, the likeability of a political candidate, how enjoyable a movie is, and so on. Analysis can be performed on the other individuals 160 so that collective mental states of the overall group can be summarized. The rendering can include displaying collective mental states from the plurality of individuals.

In one embodiment, a hundred people view several movie trailers, with facial and physiological data captured from each. The facial and physiological data can be analyzed to infer the mental states of each individual and the collective response of the group as a whole. The movie trailer which has the greatest arousal and positive valence can be considered to motivate viewers of the movie trailer to be positively pre-disposed to go see the movie when it is released. Based on the collective response, the best movie trailer can then be selected for use in advertising an upcoming movie. In some embodiments, the demographics of the individuals is used to determine which movie trailer is best suited for different viewers. For example, one movie trailer can be recommended where teenagers will be the primary audience. Another movie trailer can be recommended where the parents of the teenagers will be the primary audience. In some embodiments, webcams or other cameras are used to analyze the gender and age of people as they interact with media. Further, IP addresses can be collected indicating geography where analysis is being collected. This information and other information can be included as metadata and used as part of the analysis. For instance, teens who are up past midnight on Friday nights in an urban setting might be identified as a group for analysis.

In another embodiment, a dozen people opt in for allowing web cameras to observe facial expressions and then have physiological responses collected while they are interacting with a website for a given retailer. The mental states of each of the dozen people can be inferred based on their arousal and valence analyzed from the facial expressions and physiological responses. Certain web-page designs can be understood by the retailer to cause viewers to be more favorable to specific products and even to make a buying decision more quickly. Alternatively, web pages which cause confusion can be replaced with web pages which can cause viewers to respond with confidence.

An aggregating machine 170 can be part of the system 100. Other sources of data 172 can be provided as input to the system 100 and can be used to aid in the mental state evaluation for the individual on whom the data collection 110 was performed. The other data sources 172 can include news feeds, Facebook™ pages, Twitter™, Flickr™, and other social networking and media. The aggregating machine 170 can analyze these other data sources 172 to aid in the evaluation of the mental state of the individual on which the data was collected.

In one example embodiment, an employee of a company opts in to a self-assessment program where his or her face and electrodermal activity are monitored while performing job duties. The employee can also opt in to a tool where the aggregator 170 reads blog posts and social networking posts for mentions of the job, company, mood or health. Over time, the employee is able to review social networking presence in context of perceived feelings for that day at work. The employee can also see how his or her mood and attitude can affect what is posted. One embodiment could be non-invasive, such as just counting the number of social network posts, or as invasive as pumping the social networking content through an analysis engine that infers mental state from textual content.

In another embodiment, a company might want to understand how news stories about the company in the Wall Street Journal™ and other publications affects employee morale and job satisfaction. The aggregator 170 can be programmed to search for news stories mentioning the company and link them back to the employees participating in this experiment. A person doing additional analysis can view the news stories about the company to provide additional context to each participant's mental state.

In yet another embodiment, a facial analysis tool processes facial action units and gestures to infer mental states. As images are stored, metadata can be attached, such as the name of the person whose face is in a video that is part of the facial analysis. This video and metadata can be passed through a facial recognition engine and be taught the face of the person. Once the face is recognizable to a facial recognition engine, the aggregator 170 can spider across the Internet, or just to specific web sites such as Flickr™ and Facebook™, to find links with the same face. The additional pictures of the person located by facial recognition can be resubmitted to the facial analysis tool for an analysis to provide deeper insight into the subject's mental state.

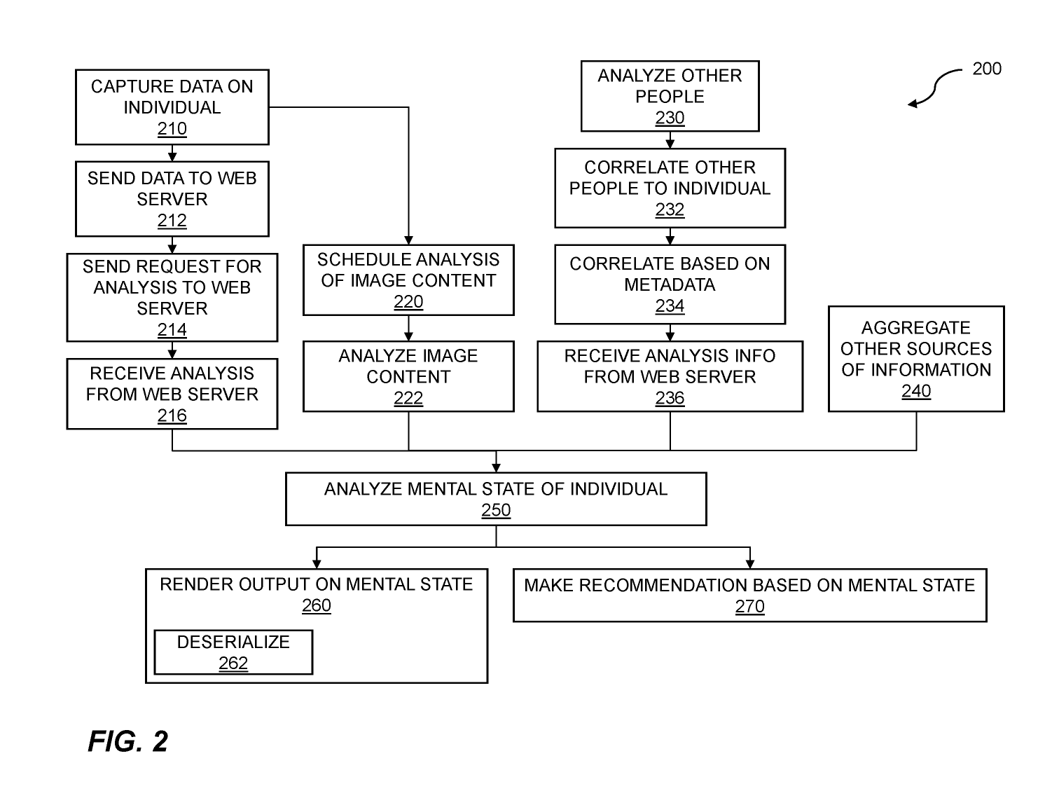

FIG. 2 is a flow diagram for obtaining and using data in mental state analysis. The flow 200 describes a computer implemented method for analyzing mental states. The flow begins by capturing data on an individual 210 into a computer system, wherein the data provides information for evaluating the mental state of the individual. The data which was captured can be correlated to an experience by the individual. The experience can comprise interacting with a web site, a movie, a movie trailer, a product, a computer game, a video game, personal game console, a cell phone, a mobile device, an advertisement, and consuming a food. “Interacting with” can refer to simply viewing, or it can mean viewing and responding. The data on the individual can further include information on hand gestures and body language. The data on the individual can include facial expressions, physiological information, and accelerometer readings. The facial expressions can further comprise head gestures. The physiological information can include electrodermal activity, skin temperature, heart rate, heart rate variability, and respiration. The physiological information can be obtained without contacting the individual, such as through analyzing facial video. The information can be captured and analyzed in real time, on a just-in-time basis, or on a scheduled analysis basis.

The flow 200 continues with sending the data which was captured to a web service 212. The sent data can include image, physiological, and accelerometer information. The data can be sent for further mental state analysis or for correlation with other people's data, or another analysis. In some embodiments, the data which is sent to the web service is a subset of the data which was captured on the individual. The web servers can be a web site, ftp site, or server which provides access to a larger group of analytical tools and data relating to mental states. The web servers can be a conduit for data that was collected on other people or from other sources of information. In some embodiments, the process includes indexing the data which was captured on a web service. The flow 200 can continue with sending a request for analysis to the web service 214. The analysis can include correlating the data which was captured with other people's data, analyzing the data which was captured for mental states, and the like. In some embodiments, the analysis is generated just in time based on a request for the analysis. The flow 200 continues with receiving analysis from the web service 216, wherein the analysis is based on the data on the individual which was captured. The received analysis can correspond to what was requested, can be based on the data captured, or can be some other logical analysis based on the mental state analysis or the data that was captured recently.

In some embodiments, the data which was captured includes images of the individual. The images can be a sequence of images and can be captured by video camera, web camera still shots, thermal imager, CCD devices, phone camera, or another camera type apparatus. The flow 200 can include scheduling analysis of the image content 220. The analysis can be performed real time, on a just-in-time basis, or scheduled for later analysis. Some of the data which was captured can require further analysis beyond what is possible in real time. Other types of data can also require further analysis and can involve scheduling analysis of a portion of the data which was captured and indexed and performing the analysis of the portion of the data which was scheduled. The flow 200 can continue with analysis of the image content 222. In some embodiments, analysis of video includes the data on facial expressions and head gestures. The facial expressions and head gestures can be recorded on video. The video can be analyzed for action units, gestures, and mental states. In some embodiments, the video analysis is used to evaluate skin pore size, which can be correlated to skin conductance or another physiological evaluation. In some embodiments, the video analysis is used to evaluate pupil dilation.

The flow 200 includes analysis of other people 230. Information from a plurality of other individuals can be analyzed, wherein the information allows evaluation of the mental state of each of the plurality of other individuals and correlates the mental state of each of the plurality of other individuals to the data which was captured and indexed on the mental state of the individual. Evaluation can also be allowed for a collective mental state of the plurality of other individuals. The other individuals can be grouped based on demographics, based on geographical locations, or based on other factors of interest in the evaluation of mental states. The analysis can include each type of data captured on the individual 210. Alternatively, analysis on the other people 230 can include other data, such as social media network information. The other people, and their associated data, can be correlated to the individual 232 on which the data was captured. The correlation can be based on common experience, common mental states, common demographics, or other factors. In some embodiments, the correlation is based on metadata 234 from the individual and metadata from the plurality of other people. The metadata can include time stamps, self-reporting results, and other information. Self-reporting results can include an indication of whether someone liked the experience they encountered, such as a video that was viewed. The flow 200 can continue with receiving analysis information from the web service 236 on a plurality of other people, wherein the information allows evaluation of the mental state of each of the plurality of other people and correlation of the mental state of each of the plurality of other people to the data which was captured on the mental state of the individual. The analysis which is received from the web service can be based on specific access rights. A web service can have data on numerous groups of individuals. In some cases, mental state analysis can only be authorized on one or more groups.

The flow 200 can include aggregating other sources of information 240 in the mental state analysis effort. The sources of information can include news feeds, Facebook™ entries, Flickr™, Twitter™ tweets, and other social networking sites. The aggregating can involve collecting information from the various sites which the individual visits or for which the individual creates content. The other sources of information can be correlated to the individual to help determine the relationship between the individual's mental states and the other sources of information.

The flow 200 continues with analysis of the mental states of the individual 250. The data which was captured, the image content which was analyzed, the correlation to the other people, and other sources of information which were aggregated, can each be used to infer one or more mental states for the individual. The data can be analyzed to produce mental state information. Further, a mental state analysis can be performed for a group of people, including the individual and one or more people from the other people. The process can include automatically inferring a mental state based on the data on the individual that was captured. The mental state can be a cognitive state. The mental state can be an emotional state. A mental state can be a combination of cognitive and affective states. A mental state can be inferred, or a mental state can be estimated along with a probability for the individual being in that mental state. The mental states that can be evaluated can include happiness, sadness, contentedness, worry, concentration, anxiety, confusion, delight, and confidence. In some embodiments, an indicator of mental state is simply tracking and analyzing smiles.

Mental states can be inferred based on physiological data, accelerometer readings, or on facial images which are captured. The mental states can be analyzed based on arousal and valence. Arousal can range from being highly activated, such as when someone is agitated, to being entirely passive, such as when someone is bored. Valence can range from being very positive, such as when someone is happy, to being very negative, such as when someone is angry. Physiological data can include electrodermal activity (EDA) or skin conductance or galvanic skin response (GSR), accelerometer readings, skin temperature, heart rate, heart rate variability, and other types of analysis of a human being. It will be understood that both here and elsewhere in this document, physiological information can be obtained either by sensor or by facial observation. In some embodiments, the facial observations are obtained with a webcam. In some instances, an elevated heart rate indicates a state of excitement. An increased level of skin conductance can correspond to being aroused. Small, frequent accelerometer movement readings can indicate fidgeting and boredom. Accelerometer readings can also be used to infer context, such as working at a computer, riding a bicycle, or playing a guitar. Facial data can include facial actions and head gestures used to infer mental states. Further, the data can include information on hand gestures or body language and body movements such as visible fidgets. In some embodiments, these movements are captured by cameras or sensor readings. Facial data can include tilting the head to the side, leaning forward, a smile, a frown, and many other gestures or expressions. Tilting of the head forward can indicate engagement with what is being shown on an electronic display. Having a furrowed brow can indicate concentration. A smile can indicate being positively disposed or being happy. Laughing can indicate that a subject has been found to be funny and enjoyable. A tilt of the head to the side and a furrow of the brows can indicate confusion. A shake of the head negatively can indicate displeasure. These and many other mental states can be indicated based on facial expressions and physiological data that is captured. In embodiments, physiological data, accelerometer readings, and facial data are each used as contributing factors in algorithms that infer various mental states. Additionally, higher complexity mental states can be inferred from multiple pieces of physiological data, facial expressions, and accelerometer readings. Further, mental states can be inferred based on physiological data, facial expressions, and accelerometer readings collected over a period of time.

The flow 200 continues with rendering an output which describes the mental state 260 of the individual based on the analysis which was received. The output can be a textual or numeric output indicating one or more mental states. The output can be a graph with a timeline of an experience and the mental states encountered during that experience. The output rendered can be a graphical representation of physiological, facial, or accelerometer data collected. Likewise, a result can be rendered which shows a mental state and the probability of the individual being in that mental state. The process can include annotating the data which was captured and rendering the annotations. The rendering can display the output on a computer screen. The rendering can include displaying arousal and valence. The rendering can store the output on a computer readable memory in the form of a file or data within a file. The rendering can be based on data which is received from the web service. Various types of data can be received including a serialized object in the form of JavaScript Object Notation (JSON) or in an XML or CSV type file. The flow 200 can include de-serializing 262 the serialized object into a form for a JavaScript object. The JavaScript object can then be used to output text or graphical representations of the mental states.

In some embodiments, the flow 200 includes recommending a course of action based on the mental state 270 of the individual. The recommending can include modifying a question queried to a focus group, changing an advertisement on a web page, editing a movie which was viewed to remove an objectionable section, changing direction of an electronic game, changing a medical consultation presentation, editing a confusing section of an internet-based tutorial, or the like.

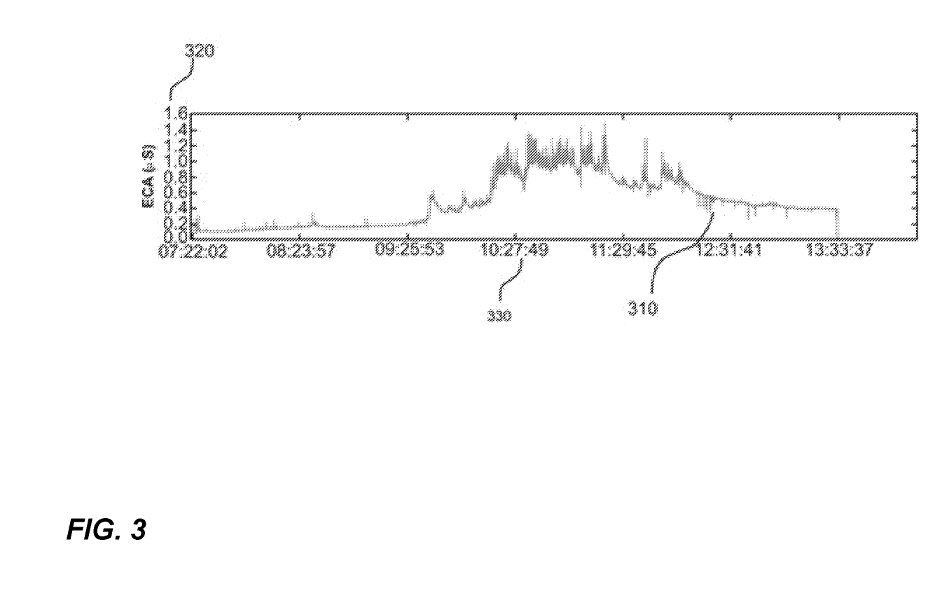

FIG. 3 is a graphical rendering of electrodermal activity. Electrodermal activity can include skin conductance which, in some embodiments, is measured in the units of micro-Siemens. A graph line 310 shows the electrodermal activity collected for an individual. The value for electrodermal activity is shown on the y-axis 320 for the graph. The electrodermal activity was collected over a period of time and the timescale 330 is shown on the x-axis of the graph. In some embodiments, electrodermal activity for multiple individuals is displayed when desired or shown on an aggregated basis. Markers can be included and can identify a section of the graph. The markers can be used to delineate a section of the graph that is or can be expanded. The expansion can cover a short period of time on which further analysis or review can be focused. This expanded portion can be rendered in another graph. Markers can also be included to identify sections corresponding to specific mental states. Each waveform or timeline can be annotated. A beginning annotation and an ending annotation can mark the beginning and end of a region or timeframe. A single annotation can mark a specific point in time. Each annotation can have associated text which was entered automatically or entered by a user. A text box can be displayed which includes the text.

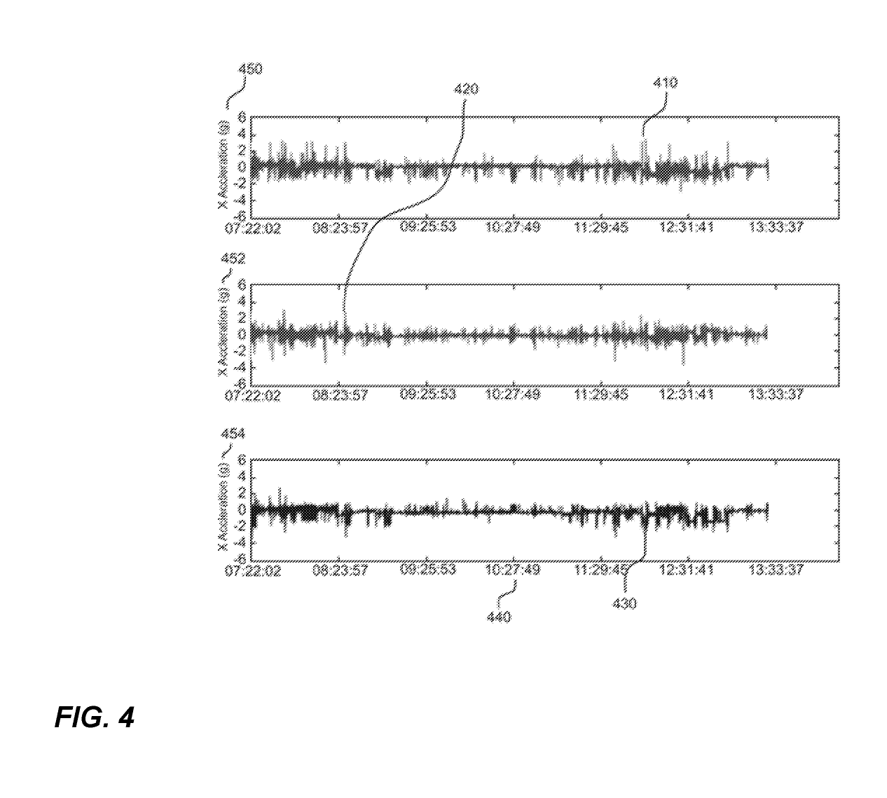

FIG. 4 is a graphical rendering of accelerometer data. One, two, or three dimensions of accelerometer data can be collected. In the example of FIG. 4, a graph for x-axis accelerometer readings is shown in a first graph 410, a graph for y-axis accelerometer readings is shown in a second graph 420, and a graph for z-axis accelerometer readings is shown in a third graph 430. The timestamps for the corresponding accelerometer readings are shown on a graph axis 440. The x acceleration values are shown on another axis 450 with the y acceleration values 452 and z acceleration values 454 shown as well. In some embodiments, accelerometer data for multiple individuals is displayed when desired or shown on an aggregated basis. Markers and annotations can be included and used similarly to those discussed in FIG. 3.

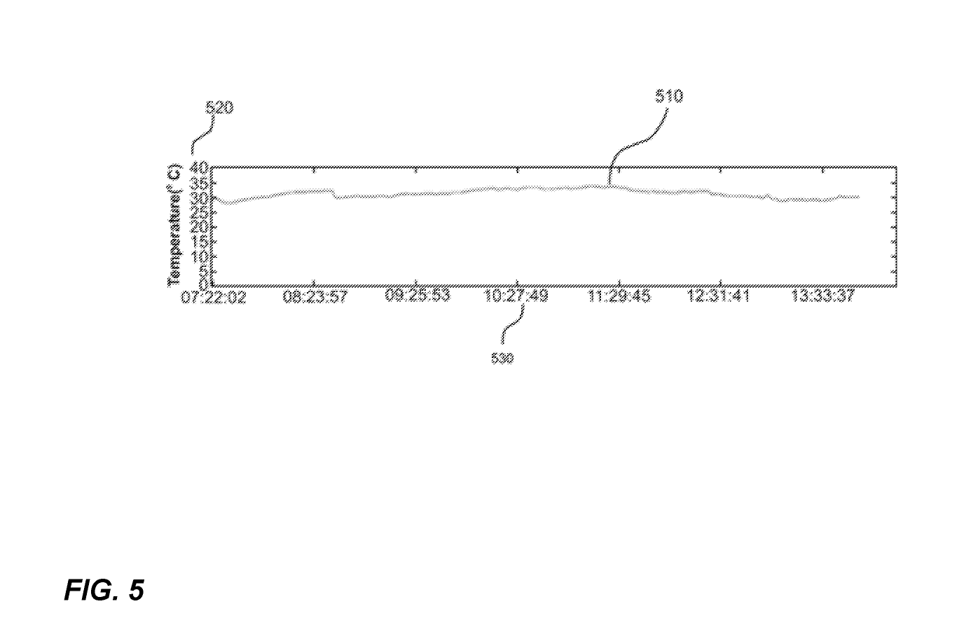

FIG. 5 is a graphical rendering of skin temperature data. A graph line 510 shows the electrodermal activity collected for an individual. The value for skin temperature is shown on the y-axis 520 for the graph. The skin temperature value was collected over a period of time and the timescale 530 is shown on the x-axis of the graph. In some embodiments, skin temperature values for multiple individuals are displayed when desired or shown on an aggregated basis. Markers and annotations can be included and used similarly to those discussed in FIG. 3.

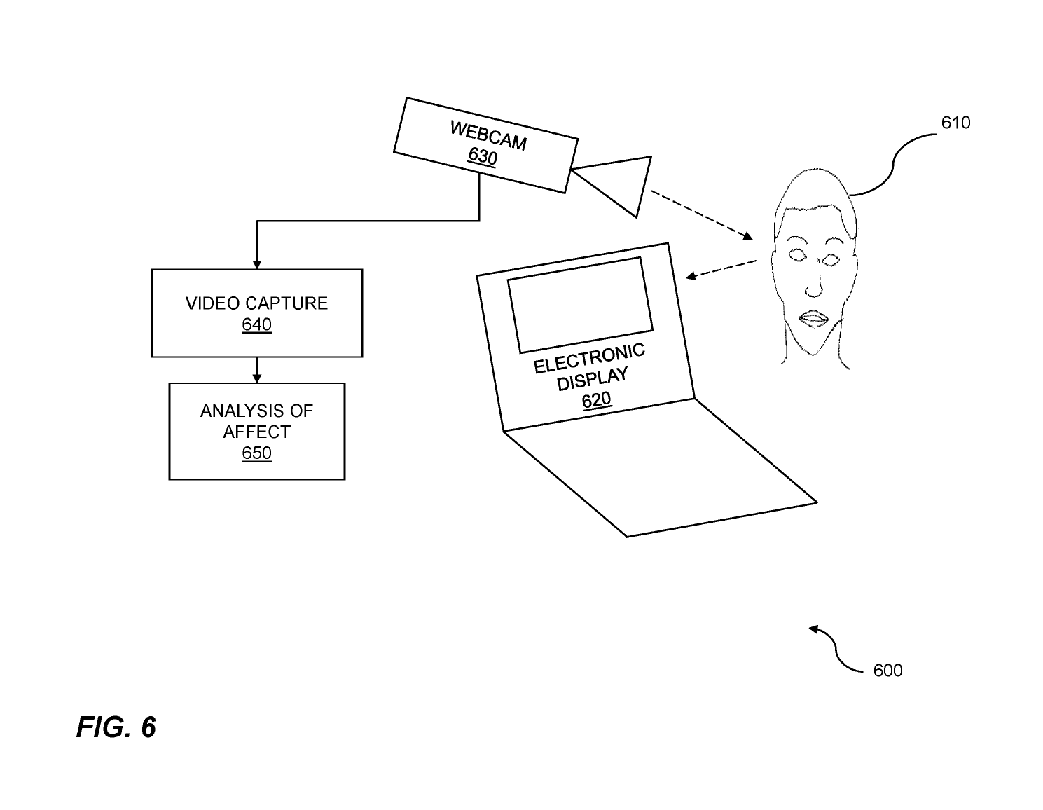

FIG. 6 shows an image collection system for facial analysis. A system 600 includes an electronic display 620 and a webcam 630. The system 600 captures facial response to the electronic display 620. In some embodiments, the system 600 captures facial responses to other stimuli such as a store display, an automobile ride, a board game, movie screen, or another experience. The facial data can include video and collection of information relating to mental states. In some embodiments, a webcam 630 captures video of the person 610. The video can be captured onto a disk, tape, into a computer system, or streamed to a server. Images or a sequence of images of the person 610 can be captured by a video camera, web camera still shots, a thermal imager, CCD devices, a phone camera, or another camera type apparatus.

The electronic display 620 can show a video or another presentation. The electronic display 620 can include a computer display, a laptop screen, a mobile device display, a cell phone display, or some other electronic display. The electronic display 620 can include a keyboard, mouse, joystick, touchpad, touch screen, wand, motion sensor, and another input means. The electronic display 620 can show a webpage, a website, a web-enabled application, or the like. The images of the person 610 can be captured by a video capture unit 640. In some embodiments, video of the person 610 is captured, while in others, a series of still images is captured. In embodiments, a webcam is used to capture the facial data.

Analysis of action units, gestures, and mental states can be accomplished using the captured images of the person 610. The action units can be used to identify smiles, frowns, and other facial indicators of mental states. In some embodiments, smiles are directly identified, and in some cases the degree of smile (small, medium, and large for example) can be identified. The gestures, including head gestures, can indicate interest or curiosity. For example, a head gesture of moving toward the electronic display 620 can indicate increased interest or a desire for clarification. Facial analysis 650 can be performed based on the information and images which are captured. The analysis can include facial analysis and analysis of head gestures. Based on the captured images, analysis of physiology can be performed. The evaluating of physiology can include evaluating heart rate, heart rate variability, respiration, perspiration, temperature, skin pore size, and other physiological characteristics by analyzing images of a person's face or body. In many cases, the evaluating can be accomplished using a webcam. Additionally, in some embodiments, physiology sensors are attached to the person to obtain further data on mental states.

The analysis can be performed in real time or “just in time”. In some embodiments, analysis is scheduled and then run through an analyzer or a computer processor which has been programmed to perform facial analysis. In some embodiments, the computer processor is aided by human intervention. The human intervention can identify mental states which the computer processor did not. In some embodiments, the processor identifies places where human intervention is useful, while in other embodiments, the human reviews the facial video and provides input even when the processor did not identify that intervention was useful. In some embodiments, the processor performs machine learning based on the human intervention. Based on the human input, the processor can learn that certain facial action units or gestures correspond to specific mental states and then can identify these mental states in an automated fashion without human intervention in the future.

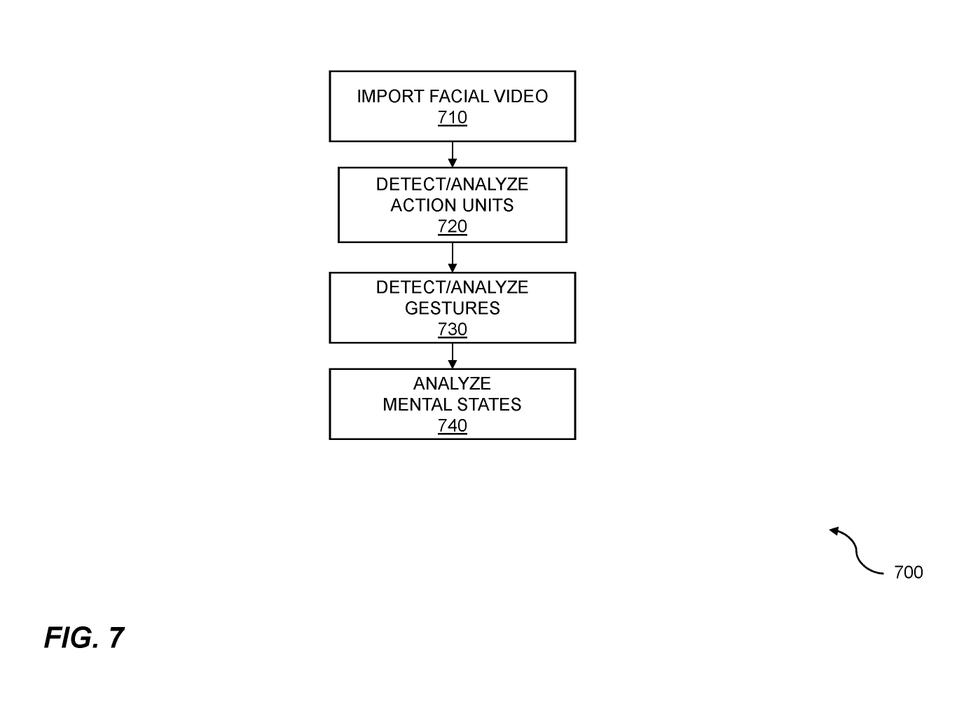

FIG. 7 is a flow diagram for performing facial analysis. Flow 700 begins with importing of facial video 710. The facial video can have been previously recorded and stored for later analysis. Alternatively, the importing of facial video can occur in real time as an individual is being observed. The flow 700 continues with action units being detected and analyzed 720. Action units can include the raising of an inner eyebrow, tightening of the lip, lowering of the brow, flaring of nostrils, squinting of the eyes, and many other possibilities. These action units can be automatically detected by a computer system analyzing the video. Alternatively, small regions of motion of the face that are not traditionally numbered on formal lists of action units can also be considered as action units for input to the analysis, such as a twitch of a smile or an upward movement above both eyes. Furthermore, a combination of automatic detection by a computer system and human input can be provided to enhance the detection of the action units or related input measures. The flow 700 continues with facial and head gestures being detected and analyzed 730. Gestures can include tilting the head to the side, leaning forward, a smile, a frown, as well as many other gestures. In the flow 100, an analysis of mental states 740 is performed. The mental states can include happiness, sadness, concentration, confusion, as well as many others. Based on the action units and facial or head gestures mental states can be analyzed, inferred, and identified.

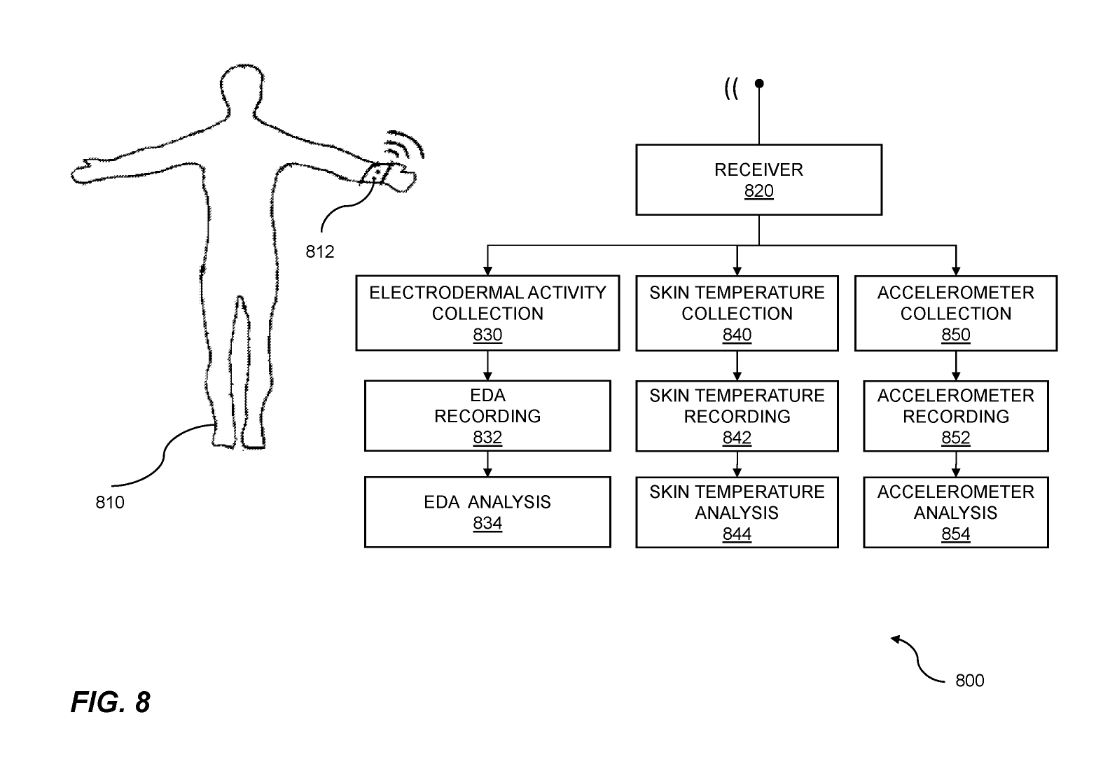

FIG. 8 is a diagram describing physiological analysis. A system 800 can analyze a person 810 for whom data is being collected. The person 810 can have a sensor 812 attached to him or her. The sensor 812 can be placed on the wrist, palm, hand, head, sternum, or another part of the body. In some embodiments, multiple sensors are placed on a person, such as on both wrists. The sensor 812 can include detectors for electrodermal activity, skin temperature, and accelerometer readings. Other detectors can also be included, such as heart rate, blood pressure, and other physiological detectors. The sensor 812 can transmit collected information to a receiver 820 using wireless technology such as Wi-Fi, Bluetooth, 802.11, cellular, or other bands. In some embodiments, the sensor 812 stores information and burst downloads the data through wireless technology. In other embodiments, the sensor 812 stores information for a later wired download. The receiver can provide the data to one or more components in the system 800. Electrodermal activity (EDA) can be collected 830. Electrodermal activity can be collected continuously, every second, four times per second, eight times per second, 32 times per second, on some other periodic basis, or based on some event. The electrodermal activity can be recorded 832. The recording can be to a disk, a tape, onto a flash drive, into a computer system, or streamed to a server. The electrodermal activity can be analyzed 834. The electrodermal activity can indicate arousal, excitement, boredom, or other mental states based on changes in skin conductance.

Skin temperature can be collected 840 continuously, every second, four times per second, eight times per second, 32 times per second, or on some other periodic basis. The skin temperature can be recorded 842. The recording can be to a disk, a tape, onto a flash drive, into a computer system, or streamed to a server. The skin temperature can be analyzed 844. The skin temperature can be used to indicate arousal, excitement, boredom, or other mental states based on changes in skin temperature.

Accelerometer data can be collected 850. The accelerometer can indicate one, two, or three dimensions of motion. The accelerometer data can be recorded 852. The recording can be to a disk, a tape, onto a flash drive, into a computer system, or streamed to a server. The accelerometer data can be analyzed 854. The accelerometer data can be used to indicate a sleep pattern, a state of high activity, a state of lethargy, or another state based on accelerometer data.

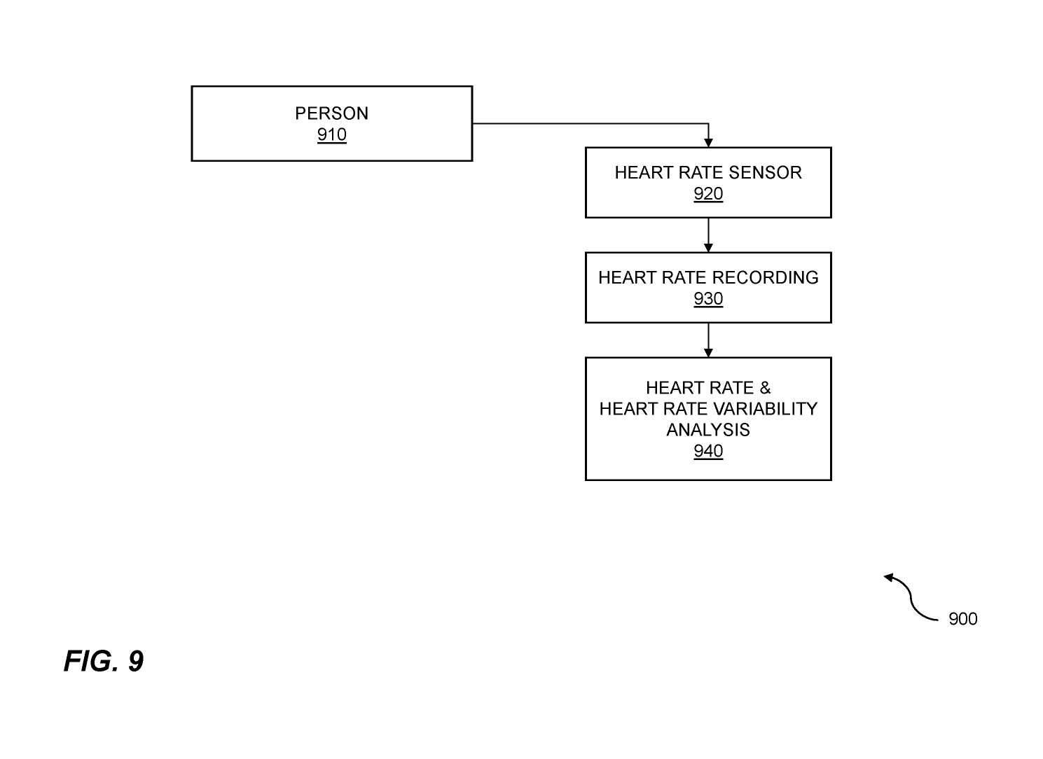

FIG. 9 is a flow diagram describing heart rate analysis. The flow 900 includes observing a person 910. The person can be observed by a heart rate sensor 920. The observation can be through a contact sensor, through video analysis which enables capture of heart rate information, or another contactless sensing. The heart rate can be recorded 930. The recording can be to a disk, a tape, onto a flash drive, into a computer system, or streamed to a server. The heart rate and heart rate variability can be analyzed 940. An elevated heart rate can indicate excitement, nervousness, or other mental states. A lowered heart rate can be used to indicate calmness, boredom, or other mental states. A heart rate being variable can indicate good health and lack of stress. A lack of heart rate variability can indicate an elevated level of stress.

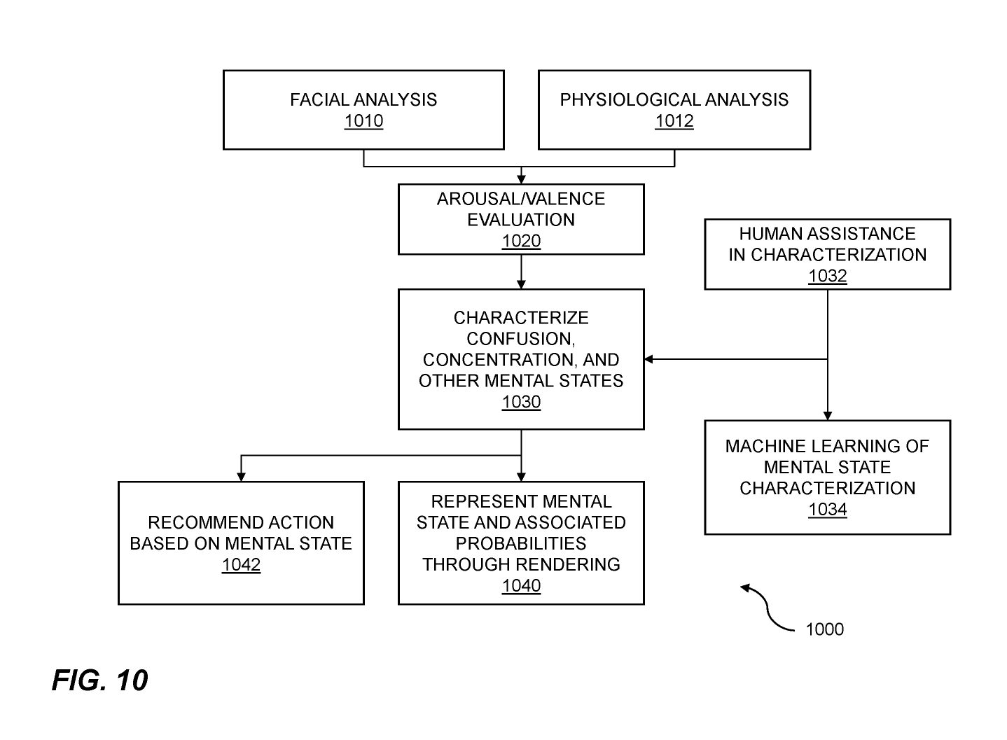

FIG. 10 is a flow diagram for performing mental state analysis and rendering. The flow 1000 can begin with various types of data collection and analysis. Facial analysis 1010 can be performed, identifying action units, facial and head gestures, smiles, and mental states. Physiological analysis 1012 can be performed. The physiological analysis can include electrodermal activity, skin temperature, accelerometer data, heart rate, and other measurements related to the human body. The physiological data can be collected through contact sensors, through video analysis, as in the case of heart rate information, or through another means. In some embodiments, an arousal and valence evaluation 1020 is performed. A level of arousal can range from being calm to being excited. A valence can be a positive or a negative predisposition. The combination of valence and arousal can be used to characterize mental states 1030, and the mental states can include confusion, concentration, happiness, contentedness, confidence, as well as other states.

In some embodiments, the characterization of mental states 1030 is completely evaluated by a computer system. In other embodiments, human assistance is provided in inferring the mental state 1032. The process can involve using a human to evaluate a portion of facial expressions, head gestures, hand gestures, or body language. A human can be used to evaluate only a small portion or even a single expression or gesture. Thus, a human can evaluate a small portion of the facial expressions, head gestures, or hand gestures. Likewise, a human can evaluate a portion of the body language of the person being observed. In embodiments, the process involves prompting a person for input on an evaluation of the mental state for a section of the data which was captured. A person can view the facial analysis or physiological analysis raw data, including video, or can view portions of the raw data or analyzed results. The person can intervene and provide input to aid in the inferring of the mental state or can identify the mental state to the computer system used in the characterization of the mental state 1030. A computer system can highlight the portions of data where human intervention is needed and can jump to the point in time where the data for that needed intervention can be presented to the human. A feedback can be provided to the person that provides assistance in characterization. Multiple people can provide assistance in characterizing mental states. Based on the automated characterization of mental states as well as evaluation by multiple people, feedback can be provided to a person to improve the her or his accuracy in characterization. Individuals can be compensated for providing assistance in characterization. Improved accuracy in characterization, based on the automated characterization or based on the other people assisting in characterization, can result in enhanced compensation.

The flow 1000 can include learning by the computer system. Machine learning of the mental state evaluation 1034 can be performed by the computer system used in the characterization of the mental state 1030. The machine learning can be based on the input from the person on the evaluation of the mental state for the section of data.

A representation of the mental state and associated probabilities can be rendered 1040. The mental state can be presented on a computer display, electronic display, cell phone display, personal digital assistance screen, or another display. The mental state can be displayed graphically. A series of mental states can be presented with the likelihood of each state for a given point in time. Likewise, a series of probabilities for each mental state can be presented over the timeline for which facial and physiological data was analyzed. In some embodiments, an action is recommended based on the mental state 1042 which was detected. An action can include recommending a question in a focus group session, changing an advertisement on a web page, editing a movie which was viewed to remove an objectionable section or boring portion, moving a display in a store, or editing a confusing section of a tutorial on the web or in a video.

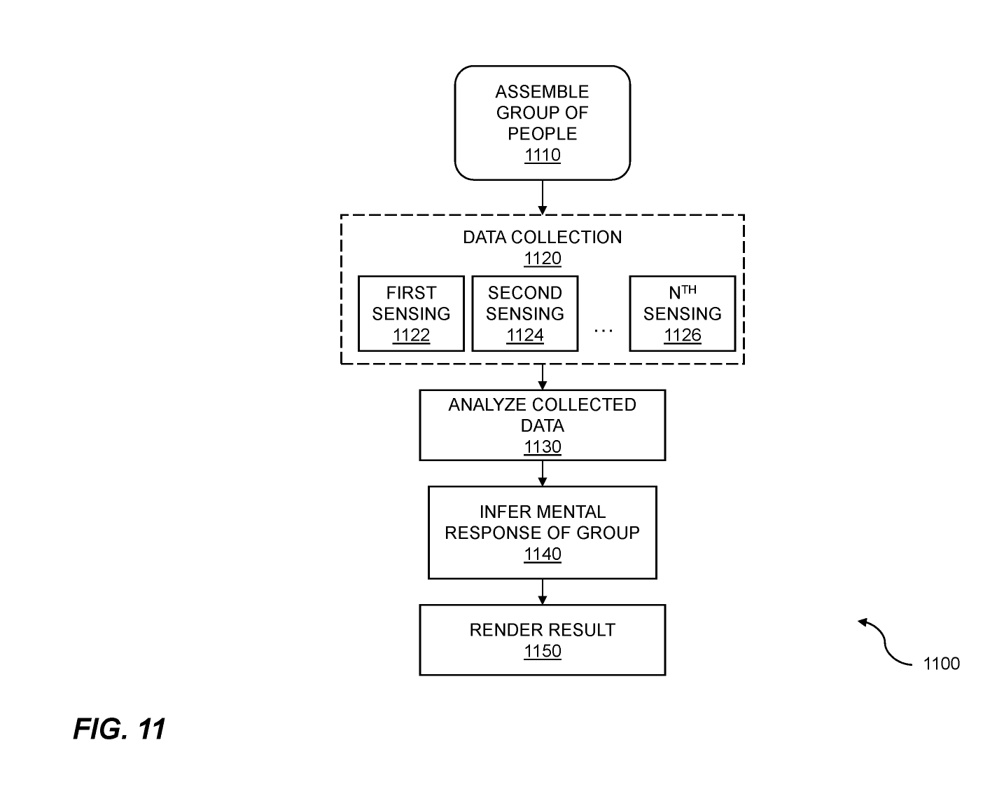

FIG. 11 is a flow diagram describing analysis of the mental response of a group. The flow 1100 can begin with assembling a group of people 1110. The group of people can have a common experience such as viewing a movie, viewing a television show, viewing a movie trailer, viewing a streaming video, viewing an advertisement, listening to a song, viewing or listening to a lecture, using a computer program, using a product, consuming a food, using a video or computer game, education through distance learning, riding in or driving a transportation vehicle such as a car, or some other experience. Data collection 1120 can be performed on each member of the group of people 1110. A plurality of sensings can occur on each member of the group of people 1110 including, for example, a first sensing 1122, a second sensing 1124, and so on through an n^{th} sensing 1126. The various sensings for which data collection 1120 is performed can include capturing facial expressions, electrodermal activity, skin temperature, accelerometer readings, heart rate, as well as other physiological information. The data which was captured can be analyzed 1130. This analysis can include characterization of arousal and valence as well as characterization of mental states for each of the individuals in the group of people 1110. The mental response of the group can be inferred 1140 providing a collective mental state. The mental states can be summarized to evaluate the common experience of all the individuals in the group of people 1110. A result can be rendered 1150. The result can be a function of time or a function of the sequence of events experienced by the people. The result can include a graphical display of the valence and arousal. The result can include a graphical display of the mental states of the individuals and the group collectively.

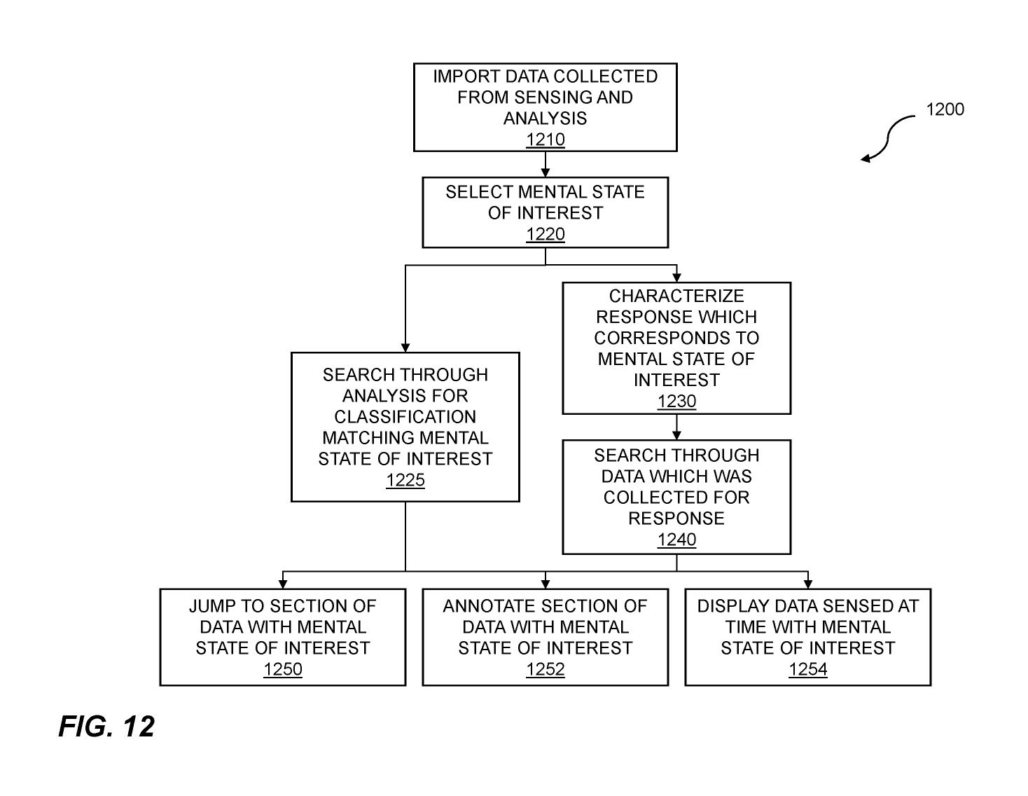

FIG. 12 is a flow diagram for identifying data portions which match a selected mental state of interest. The flow 1200 beings with an import of data collected from sensing along with any analysis performed to date 1210. The importing of data can be the loading of stored data which was previously captured or can be the loading of data which is captured in real time. The data can also already exist within the system doing the analysis. The sensing can include capture of facial expressions, electrodermal activity, skin temperature, accelerometer readings, heart rate capture, as well as other physiological information. Analysis can be performed on the various data collected, from sensing to characterizing mental states.

A mental state that interests the user can be selected 1220. The mental state of interest can be confusion, concentration, confidence, delight as well as many others. In some embodiments, analysis was previously performed on the data which was collected. The analysis can include indexing of the data and classifying mental states which were inferred or detected. When analysis has been previously performed and the mental state of interest has already been classified, a search through the analysis for one or more classifications matching the selected state can be performed 1225. By way of example, confusion can have been selected as the mental state of interest. The data which was collected can have been previously analyzed for various mental states, including confusion. When the data which was collected was indexed, a classification for confusion can have been tagged at various points in time during the data collection. The analysis can then be searched for any confusion points, as they have already been classified previously.

In some embodiments, a response is characterized which corresponds to the mental state of interest 1230. The response can be a positive valence and being aroused, as in an example where confidence is selected as the mental state of interest. The response can be reduced to valence and arousal or can be reduced further to look for action units or facial expressions and head gestures.

The data which was collected can be searched through for a response 1240 corresponding to the selected state. The sensed data can be searched, or derived analysis from the collected data can be searched. The search can look for action units, facial expressions, head gestures, or mental states which match the selected state for which the user is interested 1220.

The section of data with the mental state of interest can be jumped to 1250. For example, when confusion is selected, the data or analysis derived from the data can be shown corresponding to the point in time where confusion was exhibited. This “jump to feature” can be thought of as a fast-forward through the data to the interesting section where confusion or another selected mental state is detected. When facial video is considered, the key sections of the video which match the selected state can be displayed. In some embodiments, the section of the data with the mental state of interest is annotated 1252. Annotations can be placed along the timeline marking the data and the times with the selected state. In embodiments, the data sensed at the time with the selected state is displayed 1254. The data can include facial video. The data can also include graphical representation of electrodermal activity, skin temperature, accelerometer readouts, heart rate, and other physiological readings.

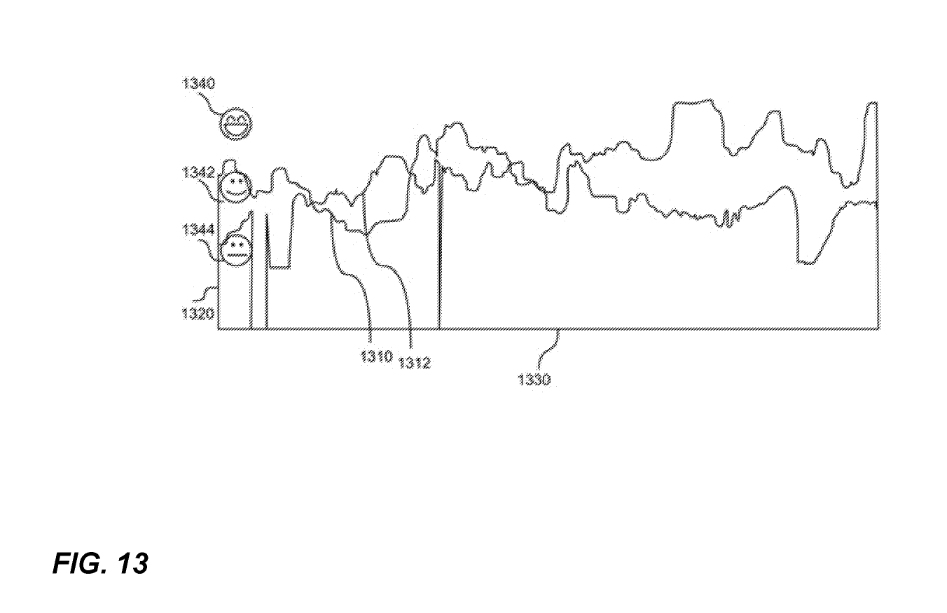

FIG. 13 is a graphical rendering of mental state analysis along with an aggregated result from a group of people. This rendering can be displayed on a web page, a web-enabled application, or another type of electronic display representation. A graph 1310 can be shown for an individual on whom affect data is collected. The mental state analysis can be based on facial image or physiological data collection. In some embodiments, the graph 1310 indicates the amount or probability of a smile being observed for the individual. A higher value or point on the graph can indicate a stronger or larger smile. In certain spots, the graph can drop out or degrade when image collection lost or was not able to identify the face of the person. The probability or intensity of an affect can be given along the y-axis 1320. A timeline can be given along the x-axis 1330. Another graph 1312 can be shown for affect collected on another individual or aggregated affect from multiple people. The aggregated information can be based on taking the average, median, or another collected value from a group of people. In some embodiments, graphical smiley face icons 1340, 1342, and 1344 are shown, providing an indication of the amount of a smile or another facial expression. A first broad smiley face icon 1340 can indicate a very large smile being observed. A second normal smiley face icon 1342 can indicate a smile being observed. A third face icon 1340 can indicate no smile. Each of the icons can correspond to a region on the y-axis 1320 that indicates the probability or intensity of a smile.

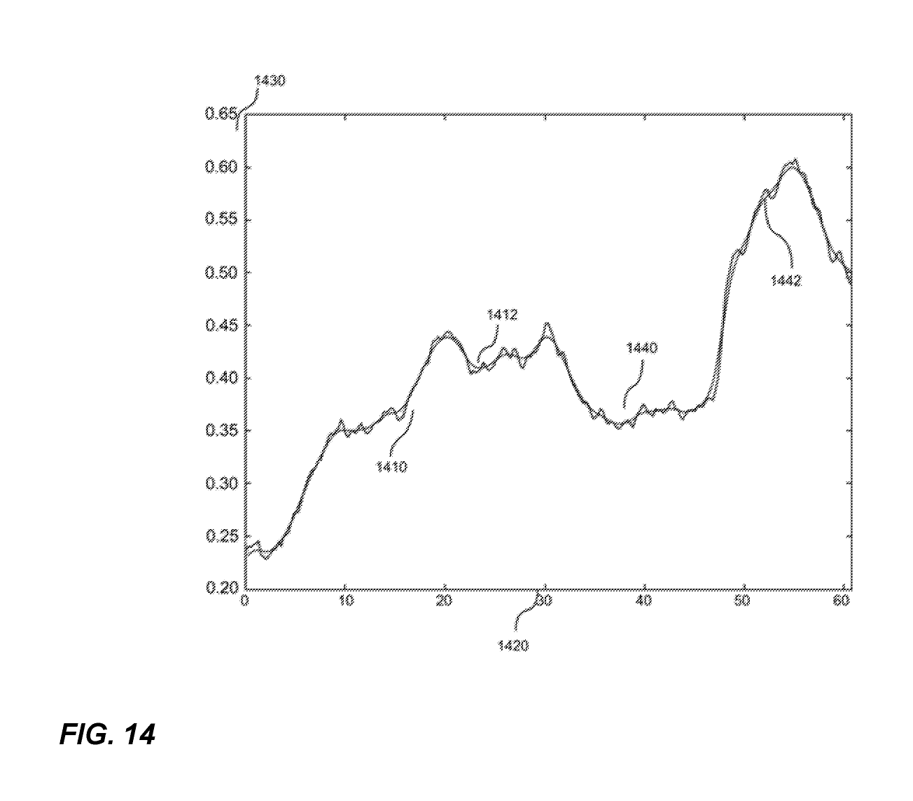

FIG. 14 is a graphical rendering of mental state analysis. This rendering can be displayed on a web page, a web-enabled application, or another type of electronic display representation. A graph 1410 can indicate the observed affect intensity or probability of occurring. A timeline can be given along the x-axis 1420. The probability or intensity of an affect can be given along the y-axis 1430. A second graph 1412 can show a smoothed version of the graph 1410. One or more valleys in the affect can be identified, such as the valley 1440. One or more peaks in affect can be identified, such as the peak 1442.

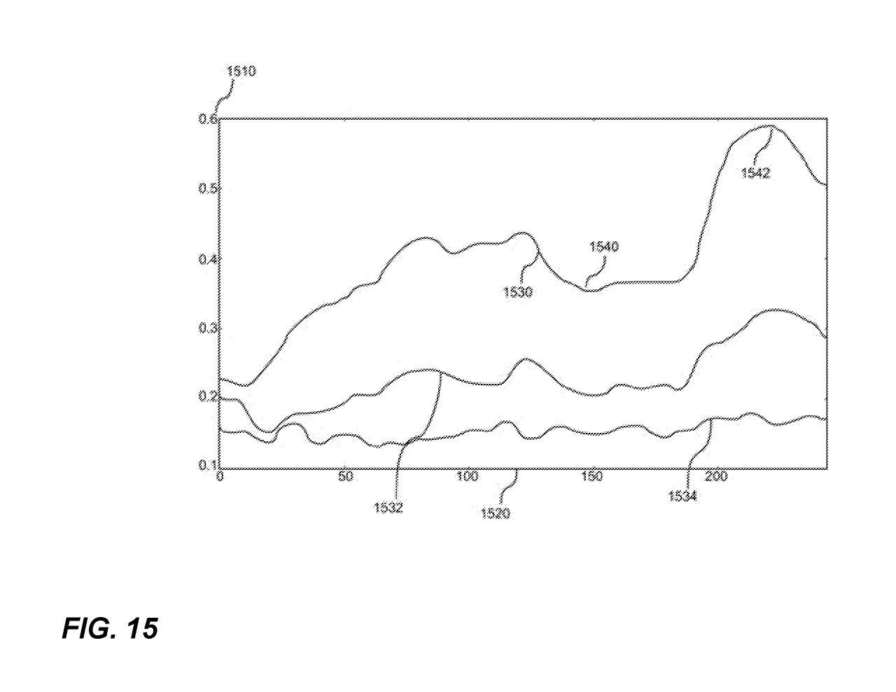

FIG. 15 is a graphical rendering of mental state analysis based on metadata. This rendering can be displayed on a web page, a web-enabled application, or another type of electronic display representation. On a graph 1510, a first line 1530, a second line 1532, and a third line 1534 can each correspond to different metadata collected. For instance, self-reporting metadata can be collected for whether the person reported that they “really liked”, “liked”, or “was ambivalent” about a certain event. The event could be a movie, a television show, a web series, a webisode, a video, a video clip, an electronic game, an advertisement, an e-book, an e-magazine, or the like. The first line 1530 can correspond to an event a person “really liked”, while the second line 1532 can correspond to another person who “liked” the event. Likewise, the third line 1534 can correspond to a different person who “was ambivalent” to the event. In some embodiments, the lines correspond to aggregated results of multiple people.

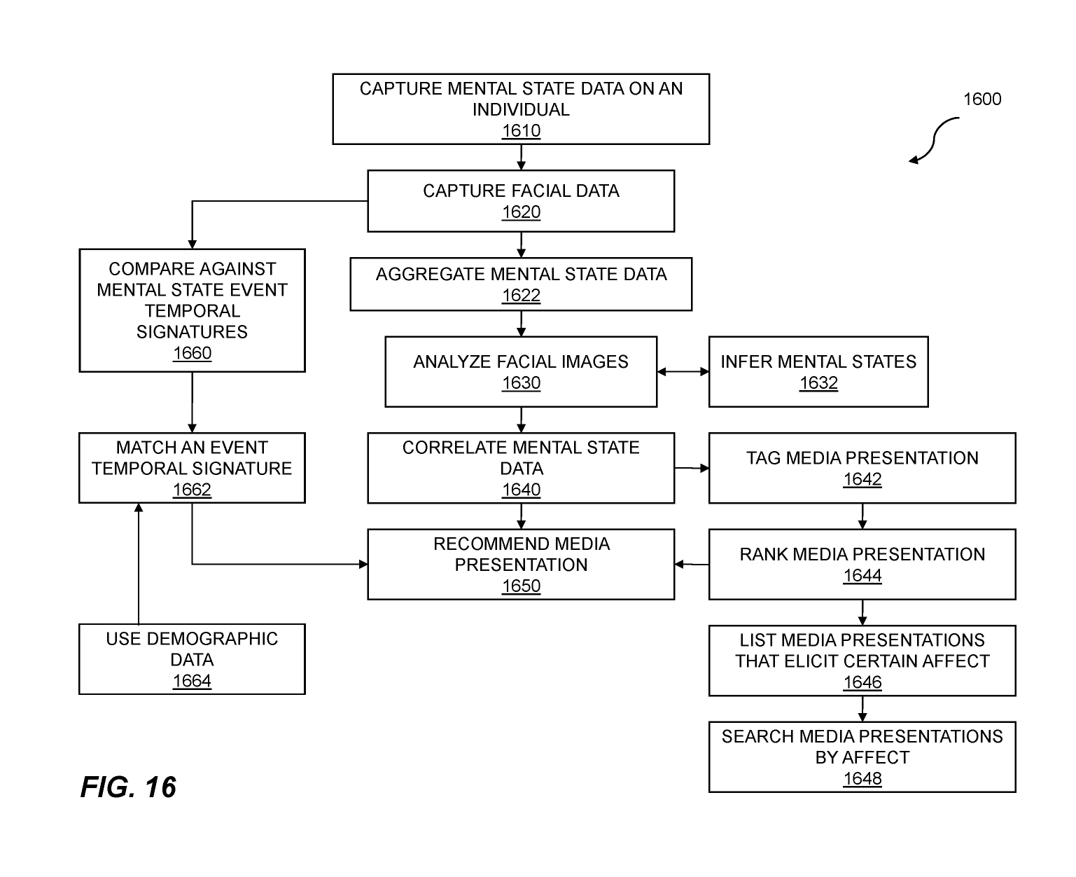

FIG. 16 is a flow diagram for affect-based recommendations. The flow 1600 describes a computer-implemented method for affect-based ranking. The flow 1600 begins with capturing mental state data on an individual 1610. The capturing can be based on displaying a plurality of media presentations to a group of people of which the individual is a part. The displaying can be done all at once or through multiple occurrences. The plurality of media presentations can include videos. The plurality of videos can include YouTube™ videos, Vimeo™ videos, or Netflix™ videos. Further, the plurality of media presentations can include a movie, a movie trailer, a television show, a web series, a webisode, a video, a video clip, an advertisement, a music video, an electronic game, an e-book, or an e-magazine. The flow 1600 continues with capturing facial data 1620. The facial data can identify a first face. The captured facial data can be from the individual or from the group of people of which the individual is a part while the plurality of media presentations is displayed. Thus, mental state data can be captured from multiple people. The affect data can include facial images. In some embodiments, the playing of the media presentations is done on a mobile device and the recording of the facial images is done with the mobile device. The flow 1600 includes aggregating the mental state data 1622 from the multiple people. The flow 1600 further includes analyzing the facial images 1630 for a facial expression. The facial expression can include a smile or a brow furrow. The flow 1600 can further comprise using the facial images to infer mental states 1632. The mental states can include frustration, confusion, disappointment, hesitation, cognitive overload, focusing, being engaged, attending, boredom, exploration, confidence, trust, delight, valence, skepticism, satisfaction, and the like.

The flow 1600 includes correlating the mental state data 1640 captured from the group of people who have viewed the plurality of media presentations and had their mental state data captured. The plurality of videos viewed by the group of people can have some common videos seen by each of the people in the group of people. In some embodiments, the plurality of videos does not include an identical set of videos. The flow 1600 can continue with tagging the plurality of media presentations 1642 with mental state information based on the mental state data which was captured. In some embodiments, the affect information is simply the affect data, while in other embodiments, the affect information is the inferred mental states. In still other embodiments, the affect information is the results of the correlation. The flow 1600 continues with ranking the media presentations 1644 relative to another media presentation based on the mental state data which was collected. The ranking can be for an individual based on the mental state data captured from the individual. The ranking can be based on anticipated preferences for the individual. In some embodiments, the ranking of a first media presentation relative to another media presentation is based on the mental state data which was aggregated from multiple people. The ranking can also be relative to media presentations previously stored with affect information. The ranking can include ranking a video relative to another video based on the mental state data which was captured. The flow 1600 can further include displaying the videos which elicit a certain affect 1646. The certain affect can include smiles, engagement, attention, interest, sadness, liking, disliking, and so on. The ranking can further comprise displaying the videos which elicited a larger number of smiles. Because of ranking, the media presentations can be sorted based on which videos are the funniest; the saddest, which generate the most tears; or on videos which engender some other response. The flow 1600 can further include searching through the videos based on a certain affect data 1648. A search 1648 can identify videos which are very engaging, funny, sad, poignant, or the like.

The flow 1600 includes comparing the mental state data that was captured for the individual against a plurality of mental state event temporal signatures 1660. In embodiments, multiple mental state event temporal signatures have been obtained from previous analysis of numerous people. The mental state event temporal signatures can include information on rise time to facial expression intensity, fall time from facial expression intensity, duration of a facial expression, and so on. In some embodiments, the mental state event temporal signatures are associated with certain demographics, ethnicities, cultures, etc. The mental state event temporal signatures can be used to identify one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, depression, envy, sympathy, embarrassment, poignancy, or mirth. The mental state event temporal signatures can be used to identify liking or satisfaction with a media presentation. The mental state event temporal signatures can be used to correlate with appreciating a second media presentation. The flow 1600 can include matching a first event signature 1662, from the plurality of mental state event temporal signatures, against the mental state data that was captured. In embodiments, an output rendering is based on the matching of the first event signature. The matching can include identifying similar aspects of the mental state event temporal signature such as rise time, fall time, duration, and so on. The matching can include matching a series of facial expressions described in mental state event temporal signatures. In some embodiments, a second mental state event temporal signature is used to identify a sequence of mental state data being expressed by an individual. In some embodiments, demographic data 1664 is used to provide a demographic basis for analyzing temporal signatures.

The flow 1600 includes recommending a second media presentation 1650 to an individual based on the affect data that was captured and based on the ranking. The recommending the second media presentation to the individual is further based on the comparing of the mental state data to the plurality of mental state event temporal signatures. The second media presentation can be a movie, a movie trailer, a television show, a web series, a webisode, a video, a video clip, an advertisement, a music video, an electronic game, an e-book, or an e-magazine. The recommending the second media presentation can be further based on the matching of the first event signature. The recommending can be based on similarity of mental states expressed. The recommending can be based on a numerically quantifiable determination of satisfaction or appreciation of the first media and an anticipated numerically quantifiable satisfaction or appreciation of second first media presentation.

Based on the mental states, recommendations to or from an individual can be provided. One or more recommendations can be made to the individual based on mental states, affect, or facial expressions. A correlation can be made between one individual and others with similar affect exhibited during multiple videos. The correlation can include a record of other videos, games, or other experiences, along with their affect. Likewise, a recommendation for a movie, video, video clip, webisode or another activity can be made to an individual based on their affect. Various steps in the flow 1600 may be changed in order, repeated, omitted, or the like without departing from the disclosed inventive concepts. Various embodiments of the flow 1600 may be included in a computer program product embodied in a non-transitory computer readable medium that includes code executable by one or more processors.

The human face provides a powerful communications medium through its ability to exhibit a myriad of expressions that can be captured and analyzed for a variety of purposes. In some cases, media producers are acutely interested in evaluating the effectiveness of message delivery by video media. Such video media includes advertisements, political messages, educational materials, television programs, movies, government service announcements, etc. Automated facial analysis can be performed on one or more video frames containing a face in order to detect facial action. Based on the detected facial action, a variety of parameters can be determined, including affect valence, spontaneous reactions, facial action units, and so on. The parameters that are determined can be used to infer or predict emotional and mental states. For example, determined valence can be used to describe the emotional reaction of a viewer to a video media presentation or another type of presentation. Positive valence provides evidence that a viewer is experiencing a favorable emotional response to the video media presentation, while negative valence provides evidence that a viewer is experiencing an unfavorable emotional response to the video media presentation. Other facial data analysis can include the determination of discrete emotional states of the viewer or viewers.

Facial data can be collected from a plurality of people using any of a variety of cameras. A camera can include a webcam, a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. In some embodiments, the person is permitted to “opt-in” to the facial data collection. For example, the person can agree to the capture of facial data using a personal device such as a mobile device or another electronic device by selecting an opt-in choice. Opting-in can then turn on the person's webcam-enabled device and can begin the capture of the person's facial data via a video feed from the webcam or other camera. The video data that is collected can include one or more persons experiencing an event. The one or more persons can be sharing a personal electronic device or can each be using one or more devices for video capture. The videos that are collected can be collected using a web-based framework. The web-based framework can be used to display the video media presentation or event as well as to collect videos from any number of viewers who are online. That is, the collection of videos can be crowdsourced from those viewers who elected to opt-in to the video data collection.

In some embodiments, a high frame rate camera is used. A high frame rate camera has a frame rate of sixty frames per second or higher. With such a frame rate, micro expressions can also be captured. Micro expressions are very brief facial expressions, lasting only a fraction of a second. They occur when a person either deliberately or unconsciously conceals a feeling.

In some cases, micro expressions happen when people have hidden their feelings from themselves (repression) or when they deliberately try to conceal their feelings from others. Sometimes the micro expressions might only last about fifty milliseconds. Hence, these expressions can go unnoticed by a human observer. However, a high frame-rate camera can be used to capture footage at a sufficient frame rate such that the footage can be analyzed for the presence of micro expressions. Micro expressions can be analyzed via action units as previously described, with various attributes such as brow raising, brow furls, eyelid raising, and the like. Thus, embodiments analyze micro expressions that are easily missed by human observers due to their transient nature.

The videos captured from the various viewers who chose to opt-in can be substantially different in terms of video quality, frame rate, etc. As a result, the facial video data can be scaled, rotated, and otherwise adjusted to improve consistency. Human factors further play into the capture of the facial video data. The facial data that is captured might or might not be relevant to the video media presentation being displayed. For example, the viewer might not be paying attention, might be fidgeting, might be distracted by an object or event near the viewer, or otherwise inattentive to the video media presentation. The behavior exhibited by the viewer can prove challenging to analyze due to viewer actions including eating, speaking to another person or persons, speaking on the phone, etc. The videos collected from the viewers might also include other artifacts that pose challenges during the analysis of the video data. The artifacts can include such items as eyeglasses (because of reflections), eye patches, jewelry, and clothing that occludes or obscures the viewer's face. Similarly, a viewer's hair or hair covering can present artifacts by obscuring the viewer's eyes and/or face.

The captured facial data can be analyzed using the facial action coding system (FACS). The FACS seeks to define groups or taxonomies of facial movements of the human face. The FACS encodes movements of individual muscles of the face, where the muscle movements often include slight, instantaneous changes in facial appearance. The FACS encoding is commonly performed by trained observers, but can also be performed on automated, computer-based systems. Analysis of the FACS encoding can be used to determine emotions of the persons whose facial data is captured in the videos. The FACS is used to encode a wide range of facial expressions that are anatomically possible for the human face. The FACS encodings include action units (AUs) and related temporal segments that are based on the captured facial expression. The AUs are open to higher order interpretation and decision-making. For example, the AUs can be used to recognize emotions experienced by the observed person. Emotion-related facial actions can be identified using the emotional facial action coding system (EMFACS) and the facial action coding system affect interpretation dictionary (FACSAID), for example. For a given emotion, specific action units can be related to the emotion. For example, the emotion of anger can be related to AUs 4, 5, 7, and 23, while happiness can be related to AUs 6 and 12. Other mappings of emotions to AUs have also been previously associated. The coding of the AUs can include an intensity scoring that ranges from A (trace) to E (maximum). The AUs can be used for analyzing images to identify patterns indicative of a particular mental and/or emotional state. The AUs range in number from 0 (neutral face) to 98 (fast up-down look). The AUs include so-called main codes (inner brow raiser, lid tightener, etc.), head movement codes (head turn left, head up, etc.), eye movement codes (eyes turned left, eyes up, etc.), visibility codes (eyes not visible, entire face not visible, etc.), and gross behavior codes (sniff, swallow, etc.). Emotion scoring can be included where intensity is evaluated as well as specific emotions, moods, or mental states.

The coding of faces identified in videos captured of people observing an event can be automated. The automated systems can detect facial AUs or discrete emotional states. The emotional states can include amusement, fear, anger, disgust, surprise, and sadness, for example. The automated systems can be based on a probability estimate from one or more classifiers, where the probabilities can correlate with an intensity of an AU or an expression. The classifiers can be used to identify into which of a set of categories a given observation can be placed. For example, the classifiers can be used to determine a probability that a given AU or expression is present in a given frame of a video. The classifiers can be used as part of a supervised machine learning technique where the machine learning technique can be trained using “known good” data. Once trained, the machine learning technique can proceed to classify new data that is captured.

The supervised machine learning models can be based on support vector machines (SVMs). An SVM can have an associated learning model that is used for data analysis and pattern analysis. For example, an SVM can be used to classify data that can be obtained from collected videos of people experiencing a media presentation. An SVM can be trained using “known good” data that is labeled as belonging to one of two categories (e.g. smile and no-smile). The SVM can build a model that assigns new data into one of the two categories. The SVM can construct one or more hyperplanes that can be used for classification. The hyperplane that has the largest distance from the nearest training point can be determined to have the best separation. The largest separation can improve the classification technique by increasing the probability that a given data point can be properly classified.

In another example, a histogram of oriented gradients (HoG) can be computed. The HoG can include feature descriptors and can be computed for one or more facial regions of interest. The regions of interest of the face can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example. The gradients can be intensity gradients and can be used to describe an appearance and a shape of a local object. The HoG descriptors can be determined by dividing an image into small, connected regions, also called cells. A histogram of gradient directions or edge orientations can be computed for pixels in the cell. Histograms can be contrast-normalized based on intensity across a portion of the image or the entire image, thus reducing any influence from illumination or shadowing changes between and among video frames. The HoG can be computed on the image or on an adjusted version of the image, where the adjustment of the image can include scaling, rotation, etc. For example, the image can be adjusted by flipping the image around a vertical line through the middle of a face in the image. The symmetry plane of the image can be determined from the tracker points and landmarks of the image.

Embodiments include identifying a first face and a second face within the facial data. Identifying and analyzing can be accomplished without further interaction with the cloud environment, in coordination with the cloud environment, and so on. In an embodiment, an automated facial analysis system identifies five facial actions or action combinations in order to detect spontaneous facial expressions for media research purposes. Based on the facial expressions that are detected, a determination can be made with regard to the effectiveness of a given video media presentation, for example. The system can detect the presence of the AUs or the combination of AUs in videos collected from a plurality of people. The facial analysis technique can be trained using a web-based framework to crowdsource videos of people as they watch online video content. The video can be streamed at a fixed frame rate to a server. Human labelers can code for the presence or absence of facial actions including symmetric smile, unilateral smile, asymmetric smile, and so on. The trained system can then be used to automatically code the facial data collected from a plurality of viewers experiencing video presentations (e.g. television programs).

Spontaneous asymmetric smiles can be detected in order to understand viewer experiences. Related literature indicates that as many asymmetric smiles occur on the right hemi face as do on the left hemi face, for spontaneous expressions. Detection can be treated as a binary classification problem, where images that contain a right asymmetric expression are used as positive (target class) samples and all other images as negative (non-target class) samples. Classifiers perform the classification, including classifiers such as support vector machines (SVM) and random forests. Random forests can include ensemble-learning methods that use multiple learning algorithms to obtain better predictive performance. Frame-by-frame detection can be performed to recognize the presence of an asymmetric expression in each frame of a video. Facial points can be detected, including the top of the mouth and the two outer eye corners. The face can be extracted, cropped and warped into a pixel image of specific dimension (e.g. 96×96 pixels). In embodiments, the inter-ocular distance and vertical scale in the pixel image are fixed. Feature extraction can be performed using computer vision software such as OpenCV™. Feature extraction can be based on the use of HoGs. HoGs can include feature descriptors and can be used to count occurrences of gradient orientation in localized portions or regions of the image. Other techniques can be used for counting occurrences of gradient orientation, including edge orientation histograms, scale-invariant feature transformation descriptors, etc. The AU recognition tasks can also be performed using Local Binary Patterns (LBP) and Local Gabor Binary Patterns (LGBP). The HoG descriptor represents the face as a distribution of intensity gradients and edge directions, and is robust in its ability to translate and scale. Differing patterns, including groupings of cells of various sizes and arranged in variously sized cell blocks, can be used. For example, 4×4 cell blocks of 8×8 pixel cells with an overlap of half of the block can be used. Histograms of channels can be used, including nine channels or bins evenly spread over 0-180 degrees. In this example, the HoG descriptor on a 96×96 image is 25 blocks×16 cells×9 bins=3600, the latter quantity representing the dimension. AU occurrences can be rendered. The videos can be grouped into demographic datasets based on nationality and/or other demographic parameters for further detailed analysis.

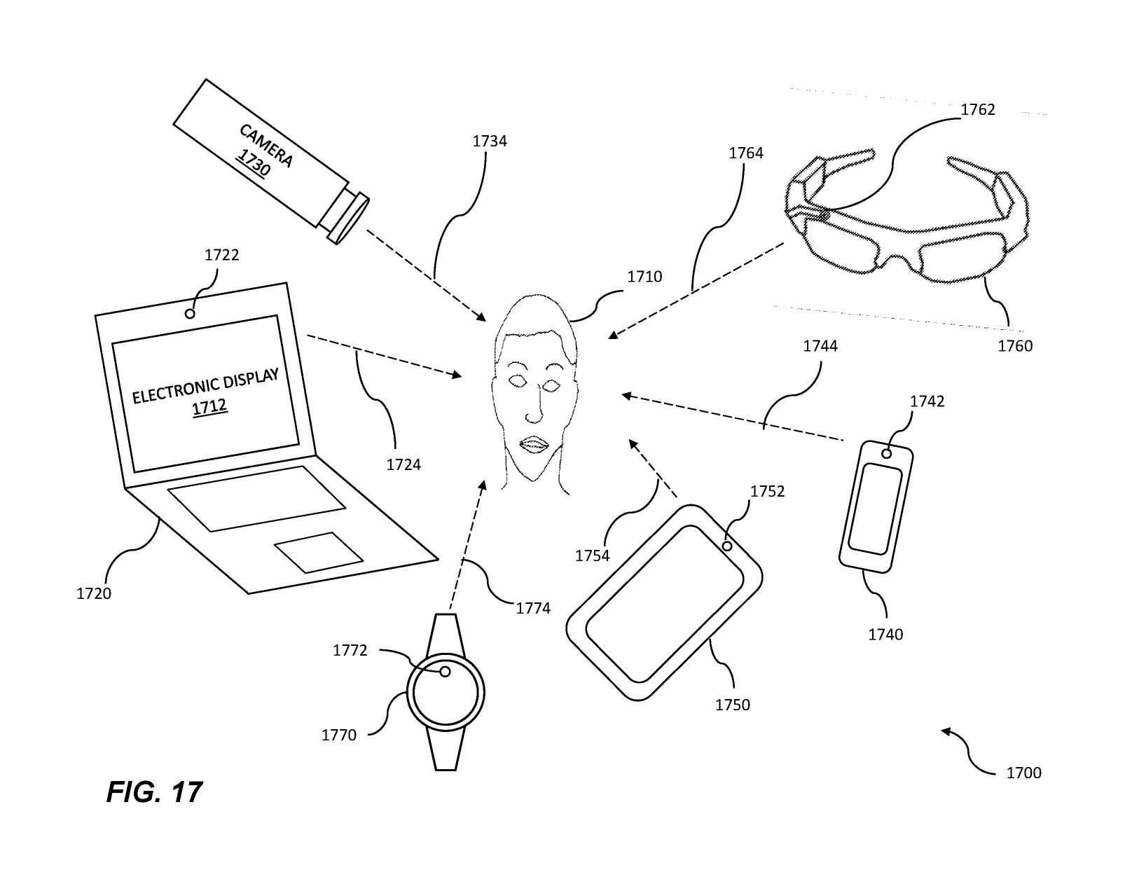

FIG. 17 shows example image collection including multiple mobile devices 1700. The images that can be collected can be analyzed to perform mental state analysis as well as to determine weights and image classifiers. The weights and the image classifiers can be used to infer an emotional metric. The multiple mobile devices can be used to collect video data on a person. While one person is shown, in practice, the video data can be collected on any number of people. A user 1710 can be observed as she or he is performing a task, experiencing an event, viewing a media presentation, and so on. The user 1710 can be viewing a media presentation or another form of displayed media. The one or more video presentations can be visible to a plurality of people instead of an individual user. If the plurality of people is viewing a media presentation, then the media presentations can be displayed on an electronic display 1712. The data collected on the user 1710 or on a plurality of users can be in the form of one or more videos. The plurality of videos can be of people who are experiencing different situations. Some example situations can include the user or plurality of users viewing one or more robots performing various tasks. The situations could also include exposure to media such as advertisements, political messages, news programs, and so on. As noted before, video data can be collected on one or more users in substantially identical or different situations. The data collected on the user 1710 can be analyzed and viewed for a variety of purposes, including expression analysis. The electronic display 1712 can be on a laptop computer 1720 as shown, a tablet computer 1750, a cell phone 1740, a television, a mobile monitor, or any other type of electronic device. In a certain embodiment, expression data is collected on a mobile device such as a cell phone 1740, a tablet computer 1750, a laptop computer 1720, or a watch 1770. Thus, the multiple sources can include at least one mobile device such as a cell phone 1740 or a tablet computer 1750, or a wearable device such as a watch 1770 or glasses 1760. A mobile device can include a forward-facing camera and/or a rear-facing camera that can be used to collect expression data. Sources of expression data can include a webcam 1722, a phone camera 1742, a tablet camera 1752, a wearable camera 1762, and a mobile camera 1730. A wearable camera can comprise various camera devices, such as the watch camera 1772.

As the user 1710 is monitored, the user 1710 might move due to the nature of the task, boredom, discomfort, distractions, or for another reason. As the user moves, the camera with a view of the user's face can change. Thus, as an example, if the user 1710 is looking in a first direction, the line of sight 1724 from the webcam 1722 is able to observe the individual's face, but if the user is looking in a second direction, the line of sight 1734 from the mobile camera 1730 is able to observe the individual's face. Further, in other embodiments, if the user is looking in a third direction, the line of sight 1744 from the phone camera 1742 is able to observe the individual's face, and if the user is looking in a fourth direction, the line of sight 1754 from the tablet camera 1752 is able to observe the individual's face. If the user is looking in a fifth direction, the line of sight 1764 from the wearable camera 1762, which can be a device such as the glasses 1760 shown and can be worn by another user or an observer, is able to observe the individual's face. If the user is looking in a sixth direction, the line of sight 1774 from the wearable watch-type device 1770 with a camera 1772 included on the device, is able to observe the individual's face. In other embodiments, the wearable device is another device, such as an earpiece with a camera, a helmet or hat with a camera, a clip-on camera attached to clothing, or any other type of wearable device with a camera or another sensor for collecting expression data. The user 1710 can also employ a wearable device including a camera for gathering contextual information and/or collecting expression data on other users. Because the user 1710 can move her or his head, the facial data can be collected intermittently when the individual is looking in a direction of a camera. In some cases, multiple people are included in the view from one or more cameras, and some embodiments include filtering out faces of one or more other people to determine whether the user 1710 is looking toward a camera. All or some of the expression data can be continuously or sporadically available from these various devices and other devices.

The captured video data can include facial expressions and can be analyzed on a computing device, such as the video capture device or on another separate device. The analysis of the video data can include the use of a classifier. For example, the video data can be captured using one of the mobile devices discussed above and sent to a server or another computing device for analysis. However, the captured video data including expressions can also be analyzed on the device which performed the capturing. For example, the analysis can be performed on a mobile device, where the videos were obtained with the mobile device and wherein the mobile device includes one or more of a laptop computer, a tablet, a PDA, a smartphone, a wearable device, and so on. In another embodiment, the analyzing comprises using a classifier on a server or other computing device other than the capturing device. The result of the analyzing can be used to infer one or more emotional metrics.

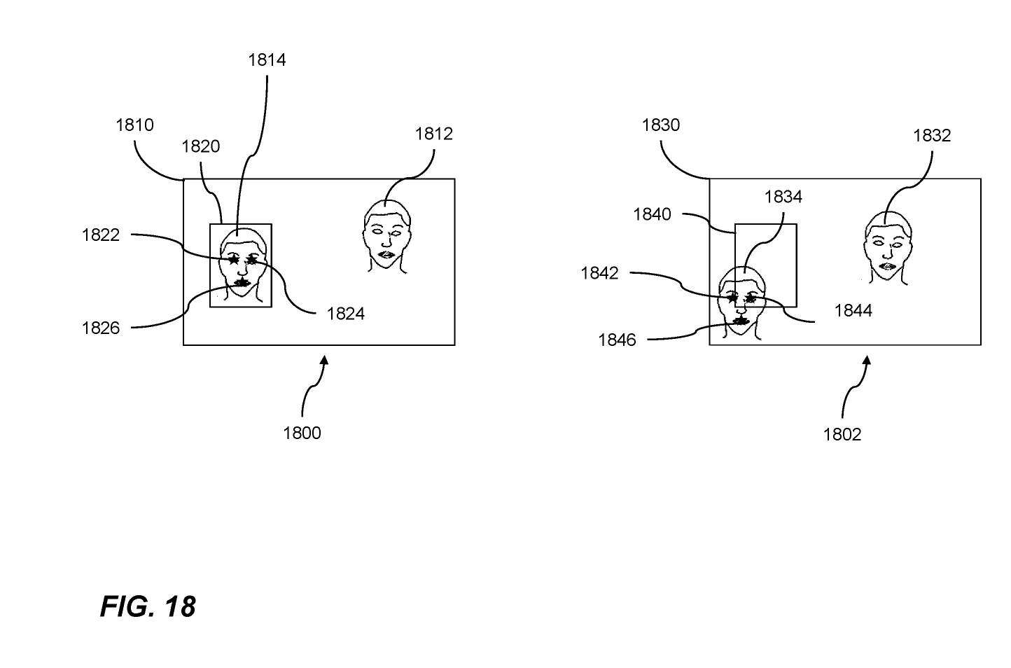

FIG. 18 illustrates feature extraction for multiple faces. One or more faces can have mental state analysis performed on them. In embodiments, features are evaluated within a deep learning environment. The feature extraction for multiple faces can be performed for faces that are detected in multiple images. The images can be analyzed to determine weights and image classifiers. The weights and the image classifiers can be used to infer an emotional metric. A plurality of images can be received of an individual viewing an electronic display. A face can be identified in an image, based on the use of classifiers. The plurality of images can be evaluated to determine mental states and/or facial expressions of the individual. The feature extraction can be performed by analysis using one or more processors, by using one or more video collection devices, and by using a server. The analysis device can be used to perform face detection for a second face, as well as for facial tracking of the first face. In embodiments, the determining weights and image classifiers is performed on a remote server based on the image data, including the first face and the second face. In other embodiments, the determining weights and image classifiers is performed on the client device based on the image data, including the first face and the second face. Other techniques can be used for determining weights and image classifiers.

One or more videos can be captured, where the videos contain one or more faces. The video or videos that contain the one or more faces can be partitioned into a plurality of frames, and the frames can be analyzed for the detection of the one or more faces. The analysis of the one or more video frames can be based on one or more classifiers. A classifier can be an algorithm, heuristic, function, or piece of code that can be used to identify into which of a set of categories a new or particular observation, sample, datum, etc., should be placed. The decision to place an observation into a category can be based on training the algorithm or piece of code or by analyzing a known set of data, known as a training set. The training set can include data for which category memberships of the data can be known. The training set can be used as part of a supervised training technique. If a training set is not available, then a clustering technique can be used to group observations into categories. The latter approach, or unsupervised learning, can be based on a measure (i.e. distance) of one or more inherent similarities among the data that is being categorized. When the new observation is received, then the classifier can be used to categorize the new observation. Classifiers can be used for many analysis applications, including analysis of one or more faces. The use of classifiers can be the basis of analyzing the one or more faces for gender, ethnicity, and age; for detection of one or more faces in one or more videos; for detection of facial features, for detection of facial landmarks, and so on. The observations can be analyzed based on one or more of a set of quantifiable properties. The properties can be described as features and explanatory variables and can include various data types such as numerical (integer-valued, real-valued), ordinal, categorical, and so on. Some classifiers can be based on a comparison between an observation and prior observations, as well as based on functions such as a similarity function, a distance function, and so on.

Classification can be based on various types of algorithms, heuristics, codes, procedures, statistics, and so on. Many techniques exist for performing classification. This classification of one or more observations into one or more groups can be based on distributions of the data values, probabilities, and so on. Classifiers can be binary, multiclass, linear, and so on. Algorithms for classification can be implemented using a variety of techniques including neural networks, kernel estimation, support vector machines, use of quadratic surfaces, and so on. Classification can be used in many application areas such as computer vision, speech and handwriting recognition, and so on. Classification can be used for biometric identification of one or more people in one or more frames of one or more videos.

Returning to FIG. 18, the detection of the first face, the second face, and multiple faces can include identifying facial landmarks, generating a bounding box, and prediction of a bounding box and landmarks for a next frame, where the next frame can be one of a plurality of frames of a video containing faces. A first video frame 1800 includes a frame boundary 1810, a first face 1812, and a second face 1814. The video frame 1800 also includes a bounding box 1820. Facial landmarks can be generated for the first face 1812. Face detection can be performed to initialize a second set of locations for a second set of facial landmarks for a second face within the video. Facial landmarks in the video frame 1800 can include the facial landmarks 1822, 1824, and 1826. The facial landmarks can include corners of a mouth, corners of eyes, eyebrow corners, the tip of the nose, nostrils, chin, the tips of ears, and so on. The performing of face detection on the second face can include performing facial landmark detection with the first frame from the video for the second face and can include estimating a second rough bounding box for the second face based on the facial landmark detection. The estimating of a second rough bounding box can include the bounding box 1820. Bounding boxes can also be estimated for one or more other faces within the boundary 1810. The bounding box can be refined, as can one or more facial landmarks. The refining of the second set of locations for the second set of facial landmarks can be based on localized information around the second set of facial landmarks. The bounding box 1820 and the facial landmarks 1822, 1824, and 1826 can be used to estimate future locations for the second set of locations for the second set of facial landmarks in a future video frame from the first video frame.

A second video frame 1802 is also shown. The second video frame 1802 includes a frame boundary 1830, a first face 1832, and a second face 1834. The second video frame 1802 also includes a bounding box 1840 and the facial landmarks 1842, 1844, and 1846. In other embodiments, multiple facial landmarks are generated and used for facial tracking of the two or more faces of a video frame, such as the shown second video frame 1802. Facial points from the first face can be distinguished from other facial points. In embodiments, the other facial points include facial points of one or more other faces. The facial points can correspond to the facial points of the second face. The distinguishing of the facial points of the first face and the facial points of the second face can be used to distinguish between the first face and the second face, to track either or both of the first face and the second face, and so on. Other facial points can correspond to the second face. As mentioned above, multiple facial points can be determined within a frame. One or more of the other facial points that are determined can correspond to a third face. The location of the bounding box 1840 can be estimated, where the estimating can be based on the location of the generated bounding box 1820 shown in the first video frame 1800. The three facial landmarks shown, facial landmarks 1842, 1844, and 1846, might lie within the bounding box 1840 or might not lie partially or completely within the bounding box 1840. For instance, the second face 1834 might have moved between the first video frame 1800 and the second video frame 1802. Based on the accuracy of the estimating of the bounding box 1840, a new estimation can be determined for a third, future frame from the video, and so on. The evaluation can be performed, all or in part, on semiconductor based logic. The evaluation can be used to infer an emotional metric.

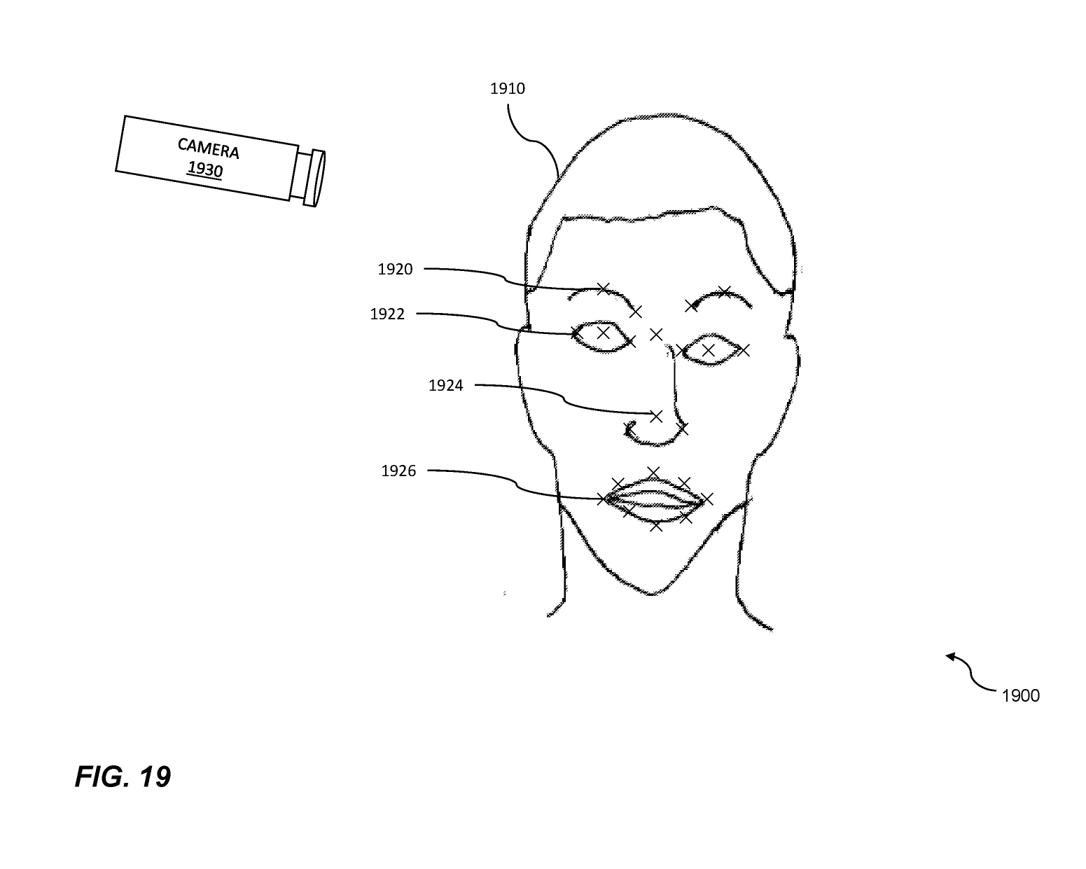

FIG. 19 shows a diagram 1900 illustrating example facial data collection including landmarks. A face 1910 can be observed using a camera 1930 in order to collect facial data that includes facial landmarks. The facial data can be collected from a plurality of people using one or more of a variety of cameras. As discussed above, the camera or cameras can include a webcam, where a webcam can include a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. The quality and usefulness of the facial data that is captured can depend, for example, on the position of the camera 1930 relative to the face 1910, the number of cameras used, the illumination of the face, etc. For example, if the face 1910 is poorly lit or over-exposed (e.g. in an area of bright light), the processing of the facial data to identify facial landmarks might be rendered more difficult. In another example, the camera 1930 being positioned to the side of the person might prevent capture of the full face. Other artifacts can degrade the capture of facial data. For example, the person's hair, prosthetic devices (e.g. glasses, an eye patch, and eye coverings), jewelry, and clothing can partially or completely occlude or obscure the person's face. Data relating to various facial landmarks can include a variety of facial features. The facial features can comprise an eyebrow 1920, an outer eye edge 1922, a nose 1924, a corner of a mouth 1926, and so on. Any number of facial landmarks can be identified from the facial data that is captured. The facial landmarks that are identified can be analyzed to identify facial action units. For example, the action units that can be identified include AU02 outer brow raiser, AU14 dimpler, AU17 chin raiser, and so on. Any number of action units can be identified. The action units can be used alone and/or in combination to infer one or more mental states and emotions. A similar process can be applied to gesture analysis (e.g. hand gestures).

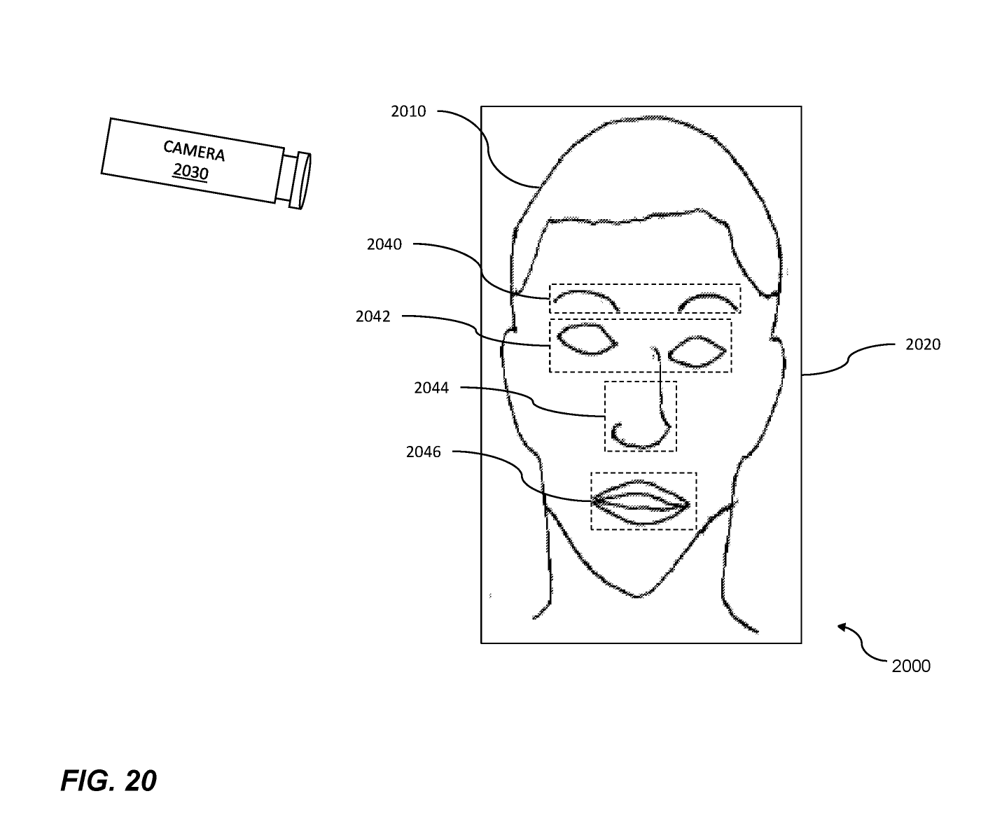

FIG. 20 shows example facial data collection including regions. The collecting of facial data, including regions, can be performed for data collected from a remote computing device. The facial data, including regions, can be collected from people as they interact with the Internet. Various regions of a face can be identified and used for a variety of purposes including facial recognition, facial analysis, and so on. The collecting of facial data, including regions, can be based on sub-sectional components of a population. The sub-sectional components can be used with performing the evaluation of content of the face, identifying facial regions, etc. The sub-sectional components can be used to provide a context. Facial analysis can be used to determine, predict, estimate, etc. mental states, emotions, and so on of a person from whom facial data can be collected. The one or more emotions that can be determined by the analysis can be represented by an image, a figure, an icon, etc. The representative icon can include an emoji. One or more emoji can be used to represent a mental state, a mood, etc. of an individual, to represent food, a geographic location, weather, and so on. The emoji can include a static image. The static image can be a predefined size such as a certain number of pixels. The emoji can include an animated image. The emoji can be based on a GIF or another animation standard. The emoji can include a cartoon representation. The cartoon representation can be any cartoon type, format, etc. that can be appropriate to representing an emoji.

In the example 2000, facial data can be collected, where the facial data can include regions of a face. The facial data that is collected can be based on sub-sectional components of a population. When more than one face can be detected in an image, facial data can be collected for one face, some faces, all faces, and so on. The facial data which can include facial regions can be collected using any of a variety of electronic hardware and software techniques. The facial data can be collected using sensors including motion sensors, infrared sensors, physiological sensors, imaging sensors, and so on. A face 2010 can be observed using a camera 2030, a sensor, a combination of cameras and/or sensors, and so on. The camera 2030 can be used to collect facial data that can be used to determine that a face is present in an image. When a face is present in an image, a bounding box 2020 can be placed around the face. Placement of the bounding box around the face can be based on detection of facial landmarks. The camera 2030 can be used to collect facial data from the bounding box 2020, where the facial data can include facial regions. The facial data can be collected from a plurality of people using any of a variety of cameras. As discussed previously, the camera or cameras can include a webcam, where a webcam can include a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. As discussed previously, the quality and usefulness of the facial data that is captured can depend on, among other examples, the position of the camera 2030 relative to the face 2010, the number of cameras and/or sensors used, the illumination of the face, any obstructions to viewing the face, and so on.

The facial regions that can be collected by the camera 2030, sensor, or combination of cameras and/or sensors can include any of a variety of facial features. The facial features in the facial regions that are collected can include eyebrows 2040, eyes 2042, a nose 2044, a mouth 2046, ears, hair, texture, tone, and so on. Multiple facial features can be included in one or more facial regions. The number of facial features that can be included in the facial regions can depend on the desired amount of data to be captured, whether a face is in profile, whether the face is partially occluded or obstructed, etc. The facial regions that can include one or more facial features can be analyzed to determine facial expressions. The analysis of the facial regions can also include determining probabilities of occurrence of one or more facial expressions. The facial features that can be analyzed can also include textures, gradients, colors, shapes, etc. The facial features can be used to determine demographic data, where the demographic data can include age, ethnicity, culture, gender, etc. Multiple textures, gradients, colors, shapes, and so on, can be detected by the camera 2030, sensor, or combination of cameras and sensors. Texture, brightness, and color, for example, can be used to detect boundaries in an image for detection of a face, facial features, facial landmarks, and so on.

A texture in a facial region can include facial characteristics, skin types, and so on. In some instances, a texture in a facial region can include smile lines, crow's feet, wrinkles, and so on. Another texture that can be used to evaluate a facial region can include a smooth portion of skin such as a smooth portion of a check. A gradient in a facial region can include values assigned to local skin texture, shading, etc. A gradient can be used to encode a texture, for instance, by computing magnitudes in a local neighborhood or portion of an image. The computed values can be compared to discrimination levels, threshold values, and so on. The gradient can be used to determine gender, facial expression, etc. A color in a facial region can include eye color, skin color, hair color, and so on. A color can be used to determine demographic data, where the demographic data can include ethnicity, culture, age, gender, etc. A shape in a facial region can include the shape of a face, eyes, nose, mouth, ears, and so on. As with color in a facial region, shape in a facial region can be used to determine demographic data including ethnicity, culture, age, gender, and so on.

The facial regions can be detected based on detection of edges, boundaries, and so on, of features that can be included in an image. The detection can be based on various types of analysis of the image. The features that can be included in the image can include one or more faces. A boundary can refer to a contour in an image plane, where the contour can represent ownership of a particular picture element (pixel) from one object, feature, etc. in the image, to another object, feature, and so on, in the image. An edge can be a distinct, low-level change of one or more features in an image. That is, an edge can be detected based on a change, including an abrupt change, in color, brightness, etc. within an image. In embodiments, image classifiers are used for the analysis. The image classifiers can include algorithms, heuristics, and so on, and can be implemented using functions, classes, subroutines, code segments, etc. The classifiers can be used to detect facial regions, facial features, and so on. As discussed above, the classifiers can be used to detect textures, gradients, color, shapes, edges, etc. Any classifier can be used for the analysis including but not limited to density estimation, support vector machines (SVM), logistic regression, classification trees, and so on. By way of example, consider facial features that can include the eyebrows 2040. One or more classifiers can be used to analyze the facial regions that can include the eyebrows to determine a probability for either a presence or an absence of an eyebrow furrow. The probability can include a posterior probability, a conditional probability, and so on. The probabilities can be based on Bayesian Statistics or another statistical analysis technique. The presence of an eyebrow furrow can indicate that the person from whom the facial data is collected is annoyed, confused, unhappy, and so on. In another example, consider facial features that can include a mouth 2046. One or more classifiers can be used to analyze the facial region that can include the mouth to determine a probability for either a presence or an absence of mouth edges turned up to form a smile. Multiple classifiers can be used to determine one or more facial expressions.

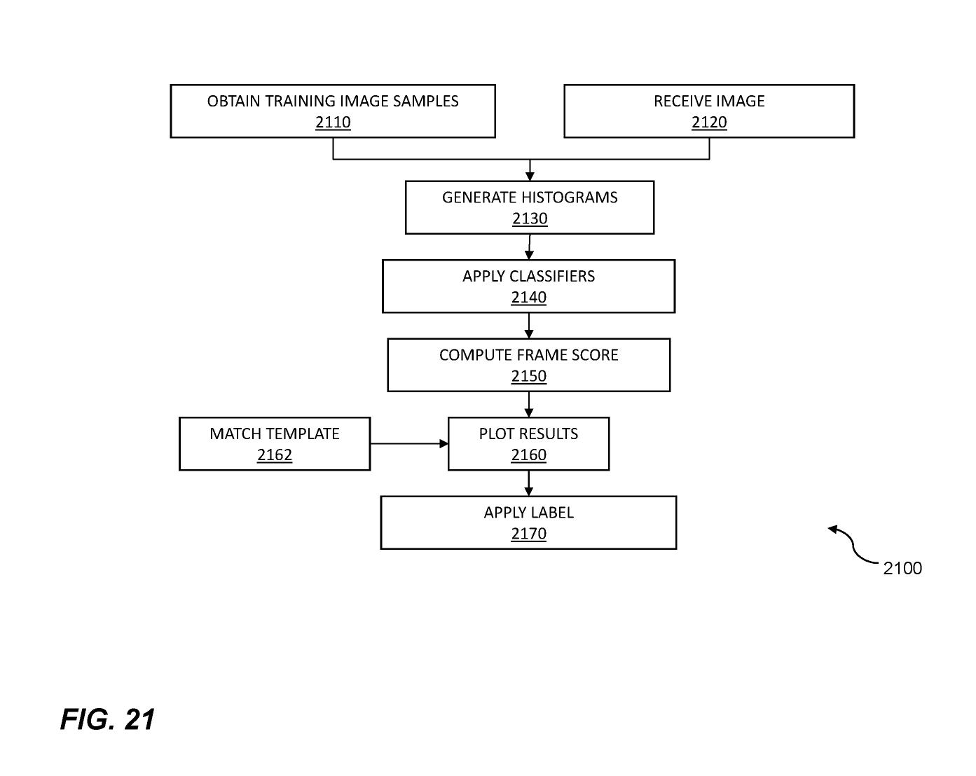

FIG. 21 is a flow diagram for detecting facial expressions. The flow 2100 can be used to automatically detect a wide range of facial expressions. A facial expression can produce strong emotional signals that can indicate valence and discrete emotional states. The discrete emotional states can include contempt, doubt, defiance, happiness, fear, anxiety, and so on. The detection of facial expressions can be based on the location of facial landmarks. The detection of facial expressions can be based on determination of action units (AU) where the action units are determined using FACS coding. The AUs can be used singly or in combination to identify facial expressions. Based on the facial landmarks, one or more AUs can be identified by number and intensity. For example, AU12 can be used to code a lip corner puller and can be used to infer a smirk.

The flow 2100 begins by obtaining training image samples 2110. The image samples can include a plurality of images of one or more people. Human coders who are trained to correctly identify AU codes based on the FACS can code the images. The training or “known good” images can be used as a basis for training a machine learning technique. Once trained, the machine learning technique can be used to identify AUs in other images that are collected using a camera, such as the camera 230 from FIG. 2, for example. The flow 2100 continues with receiving an image 2120. The image 2120 can be received from a camera, such as the camera 230 from FIG. 2, for example. As discussed above, the camera or cameras can include a webcam, where a webcam can include a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. The image 2120 that is received can be manipulated in order to improve the processing of the image. For example, the image can be cropped, scaled, stretched, rotated, flipped, etc. in order to obtain a resulting image that can be analyzed more efficiently. Multiple versions of the same image can be analyzed. For example, the manipulated image and a flipped or mirrored version of the manipulated image can be analyzed alone and/or in combination to improve analysis. The flow 2100 continues with generating histograms 2130 for the training images and the one or more versions of the received image. The histograms can be generated for one or more versions of the manipulated received image. The histograms can be based on a HoG or another histogram. As described above, the HoG can include feature descriptors and can be computed for one or more regions of interest in the training images and the one or more received images. The regions of interest in the images can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example.

The flow 2100 continues with applying classifiers 2140 to the histograms. The classifiers can be used to estimate probabilities where the probabilities can correlate with an intensity of an AU or an expression. The choice of classifiers used is based on the training of a supervised learning technique to identify facial expressions, in some embodiments. The classifiers can be used to identify into which of a set of categories a given observation can be placed. For example, the classifiers can be used to determine a probability that a given AU or expression is present in a given image or frame of a video. In various embodiments, the one or more AUs that are present include AU01 inner brow raiser, AU12 lip corner puller, AU38 nostril dilator, and so on. In practice, the presence or absence of any number of AUs can be determined. The flow 2100 continues with computing a frame score 2150. The score computed for an image, where the image can be a frame from a video, can be used to determine the presence of a facial expression in the image or video frame. The score can be based on one or more versions of the image 2120 or manipulated image. For example, the score can be based on a comparison of the manipulated image to a flipped or mirrored version of the manipulated image. The score can be used to predict a likelihood that one or more facial expressions are present in the image. The likelihood can be based on computing a difference between the outputs of a classifier used on the manipulated image and on the flipped or mirrored image, for example. The classifier that is used can be used to identify symmetrical facial expressions (e.g. smile), asymmetrical facial expressions (e.g. outer brow raiser), and so on.

The flow 2100 continues with plotting results 2160. The results that are plotted can include one or more scores for one or more frames computed over a given time t. For example, the plotted results can include classifier probability results from analysis of HoGs for a sequence of images and video frames. The plotted results can be matched with a template 2162. The template can be temporal and can be represented by a centered box function or another function. A best fit with one or more templates can be found by computing a minimum error. Other best-fit techniques can include polynomial curve fitting, geometric curve fitting, and so on. The flow 2100 continues with applying a label 2170. The label can be used to indicate that a particular facial expression has been detected in the one or more images or video frames which constitute the image 2120. For example, the label can be used to indicate that any of a range of facial expressions has been detected, including a smile, an asymmetric smile, a frown, and so on. Various steps in the flow 2100 may be changed in order, repeated, omitted, or the like without departing from the disclosed concepts. Various embodiments of the flow 2100 may be included in a computer program product embodied in a non-transitory computer readable medium that includes code executable by one or more processors.

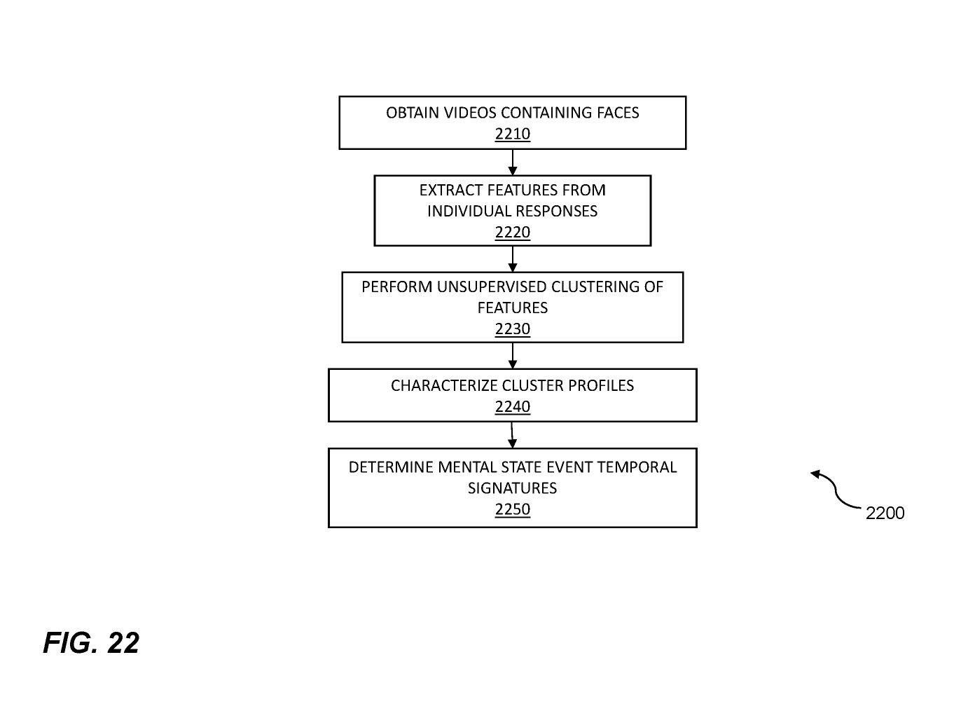

FIG. 22 is a flow for large-scale clustering of facial events. As discussed above, collection of facial video data from one or more people can include a web-based framework. The web-based framework can be used to collect facial video data from, for example, large numbers of people located over a wide geographic area. The web-based framework can include an opt-in feature that allows people to agree to facial data collection. The web-based framework can be used to render and display data to one or more people and can collect data from the one or more people. For example, the facial data collection can be based on showing one or more viewers a video media presentation through a website. The web-based framework can be used to display the video media presentation or event and to collect videos from any number of viewers who are online. That is, the collection of videos can be crowdsourced from those viewers who elected to opt-in to the video data collection. The video event can be a commercial, a political ad, an educational segment, and so on. The flow 2200 begins with obtaining videos containing faces 2210. The videos can be obtained using one or more cameras, where the cameras can include a webcam coupled to one or more devices employed by the one or more people using the web-based framework. The flow 2200 continues with extracting features from the individual responses 2220. The individual responses can include videos containing faces observed by the one or more webcams. The features that are extracted can include facial features such as an eyebrow, a nostril, an eye edge, a mouth edge, and so on. The feature extraction can be based on facial coding classifiers, where the facial coding classifiers output a probability that a specified facial action has been detected in a given video frame. The flow 2200 continues with performing unsupervised clustering of features 2230. The unsupervised clustering can be based on an event. The features can be extracted from compared mental state data. The unsupervised clustering can be based on a K-Means, where the K of the K-Means can be computed using a Bayesian Information Criterion (BICk), for example, to determine the smallest value of K that meets system requirements. Any other criterion for K can be used. The K-Means clustering technique can be used to group one or more events into various respective categories. The flow 2200 includes characterizing cluster profiles 2240. The profiles can include a variety of facial expressions such as smiles, asymmetric smiles, eyebrow raisers, eyebrow lowerers, etc. The profiles can be related to a given event. For example, a humorous video can be displayed in the web-based framework and the video data of people who have opted-in can be collected. The characterization of the collected and analyzed video can depend in part on the number of smiles that occurred at various points throughout the humorous video. Similarly, the characterization can be performed on collected and analyzed videos of people viewing a news presentation. The characterized cluster profiles can be further analyzed based on demographic data. For example, the number of smiles resulting from people viewing a humorous video can be compared to various demographic groups, where the groups can be formed based on geographic location, age, ethnicity, gender, and so on.

The flow 2200 can include determining mental state event temporal signatures 2250. The mental state event temporal signatures can include information on rise time to facial expression intensity, fall time from facial expression intensity, duration of a facial expression, and so on. In some embodiments, the mental state event temporal signatures are associated with certain demographics, ethnicities, cultures, etc. The mental state event temporal signatures can be used to identify one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, depression, envy, sympathy, embarrassment, poignancy, or mirth. Various steps in the flow 2200 may be changed in order, repeated, omitted, or the like without departing from the disclosed concepts. Various embodiments of the flow 2200 may be included in a computer program product embodied in a non-transitory computer readable medium that includes code executable by one or more processors.

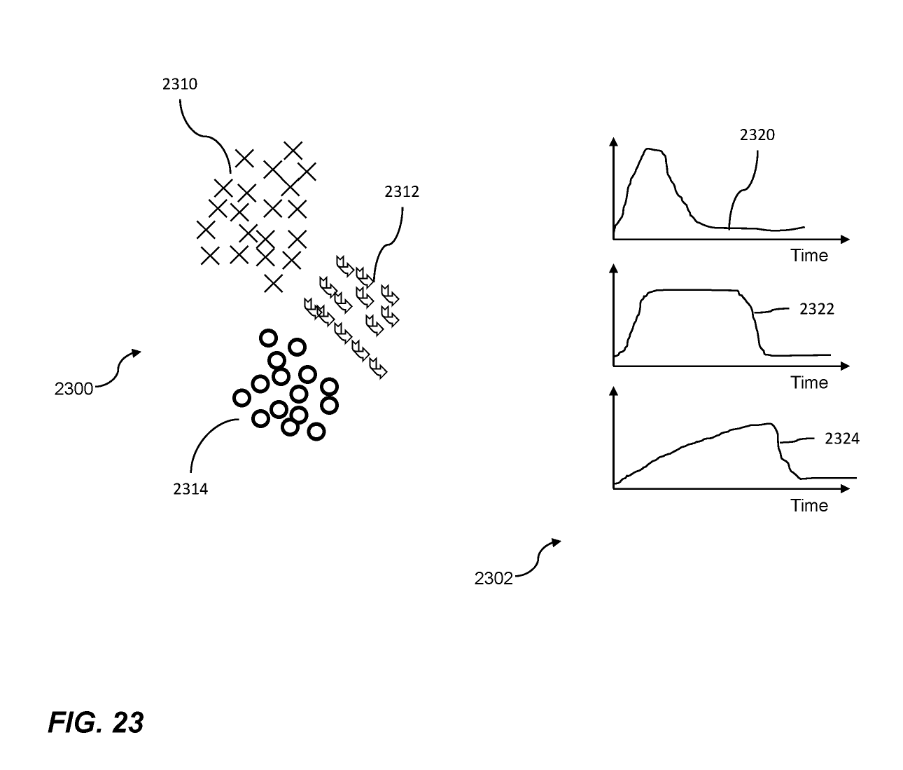

FIG. 23 shows example unsupervised clustering of features and characterization of cluster profiles. This clustering can be used to generate or identify mental state event temporal signatures. Features including samples of facial data can be clustered using unsupervised clustering. Various clusters can be formed, which include similar groupings of facial data observations. The example 2300 shows three clusters: clusters 2310, 2312, and 2314. The clusters can be based on video collected from people who have opted-in to video collection. When the data collected is captured using a web-based framework, then the data collection can be performed on a grand scale, including hundreds, thousands, or even more participants who can be located locally and/or across a wide geographic area. Unsupervised clustering is a technique that can be used to process the large amounts of captured facial data and to identify groupings of similar observations. The unsupervised clustering can also be used to characterize the groups of similar observations. The characterizations can include identifying behaviors of the participants. The characterizations can be based on identifying facial expressions and facial action units of the participants. Some behaviors and facial expressions can include faster or slower onsets, faster or slower offsets, longer or shorter durations, etc. The onsets, offsets, and durations can all correlate to time. The data clustering that results from the unsupervised clustering can support data labeling. The labeling can include FACS coding. The clusters can be partially or totally based on a facial expression resulting from participants viewing a video presentation, where the video presentation can be an advertisement, a political message, educational material, a public service announcement, and so on. The clusters can be correlated with demographic information, where the demographic information can include educational level, geographic location, age, gender, income level, and so on.

Cluster profiles 2302 can be generated based on the clusters that can be formed from unsupervised clustering, with time shown on the x-axis and intensity or frequency shown on the y-axis. The cluster profiles can be based on captured facial data, including facial expressions, for example. The facial data can include information on facial expressions, action units, head gestures, smiles, squints, lowered eyebrows, raised eyebrows, smirks, and attention. The cluster profile 2320 can be based on the cluster 2310, the cluster profile 2322 can be based on the cluster 2312, and the cluster profile 2324 can be based on the cluster 2314. The cluster profiles 2320, 2322, and 2324 can be based on smiles, smirks, frowns, or any other facial expression. Emotional states of the people who have opted-in to video collection can be inferred by analyzing the clustered facial expression data. The cluster profiles can be plotted with respect to time and can show a rate of onset, a duration, and an offset (rate of decay). Other time-related factors can be included in the cluster profiles. The cluster profiles can be correlated with demographic information as described above.

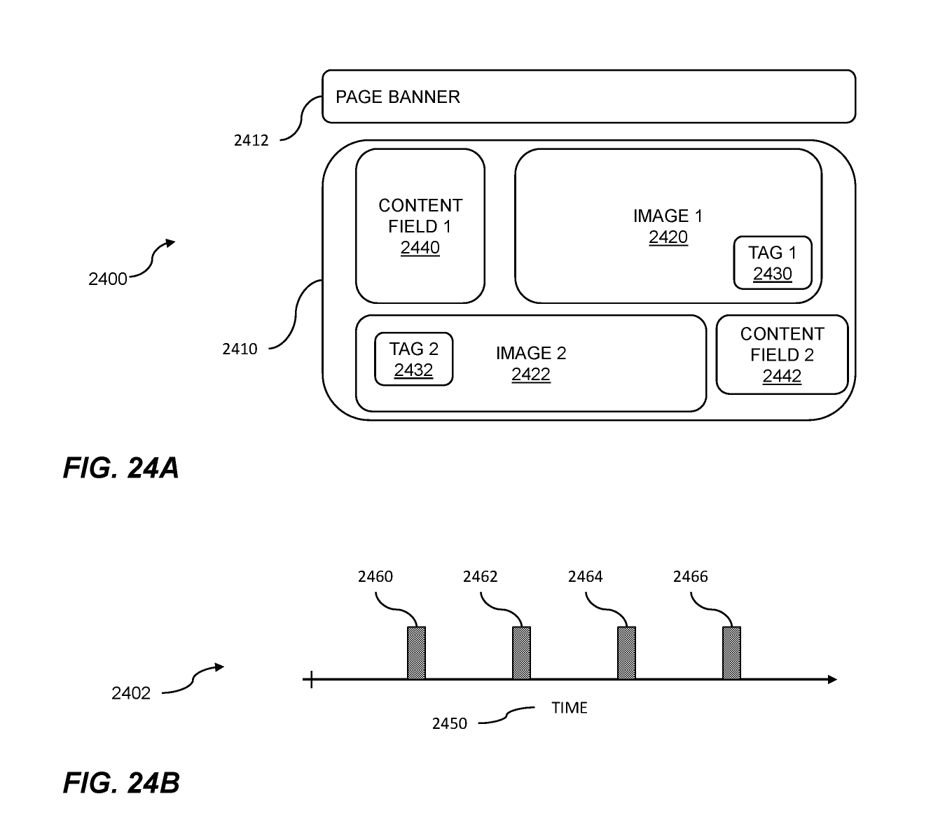

FIG. 24A shows example tags embedded in a webpage. A webpage 2400 can include a page body 2410, a page banner 2412, and so on. The page body can include one or more objects, where the objects can include text, images, videos, audio, and so on. The example page body 2410 shown includes a first image, image 1 2420; a second image, image 2 2422; a first content field, content field 1 2440; and a second content field, content field 2 2442. In practice, the page body 2410 can contain any number of images and content fields and can include one or more videos, one or more audio presentations, and so on. The page body can include embedded tags, such as tag 1 2430 and tag 2 2432. In the example shown, tag 1 2430 is embedded in image 1 2420, and tag 2 2432 is embedded in image 2 2422. In embodiments, any number of tags are imbedded. Tags can also be imbedded in content fields, in videos, in audio presentations, etc. When a user mouses over a tag or clicks on an object associated with a tag, the tag can be invoked. For example, when the user mouses over tag 1 2430, tag 1 2430 can then be invoked. Invoking tag 1 2430 can include enabling a camera coupled to a user's device and capturing one or more images of the user as the user views a media presentation (or digital experience). In a similar manner, when the user mouses over tag 2 2432, tag 2 2432 can be invoked. Invoking tag 2 2432 can also include enabling the camera and capturing images of the user. In other embodiments, other actions are taken based on invocation of the one or more tags. For example, invoking an embedded tag can initiate an analysis technique, post to social media, award the user a coupon or another prize, initiate mental state analysis, perform emotion analysis, and so on.

FIG. 24B shows example tag invoking for the collection of images. As stated above, a media presentation can be a video, a webpage, and so on. A video 2402 can include one or more embedded tags, such as a tag 2460, another tag 2462, a third tag 2464, a fourth tag 2466, and so on. In practice, any number of tags can be included in the media presentation. The one or more tags can be invoked during the media presentation. The collection of the invoked tags can occur over time as represented by a timeline 2450. When a tag is encountered in the media presentation, the tag can be invoked. For example, when the tag 2460 is encountered, invoking the tag can enable a camera coupled to a user's device and can capture one or more images of the user viewing the media presentation. Invoking a tag can depend on opt-in by the user. For example, if a user has agreed to participate in a study by indicating an opt-in, then the camera coupled to the user's device can be enabled and one or more images of the user can be captured. If the user has not agreed to participate in the study and has not indicated an opt-in, then invoking the tag 2460 does not enable the camera nor capture images of the user during the media presentation. The user can indicate an opt-in for certain types of participation, where opting-in can be dependent on specific content in the media presentation. For example, the user could opt-in to participation in a study of political campaign messages and not opt-in for a particular advertisement study. In this case, tags that are related to political campaign messages and that enable the camera and image capture when invoked would be embedded in the media presentation. However, tags imbedded in the media presentation that are related to advertisements would not enable the camera when invoked. Various other situations of tag invocation are possible.

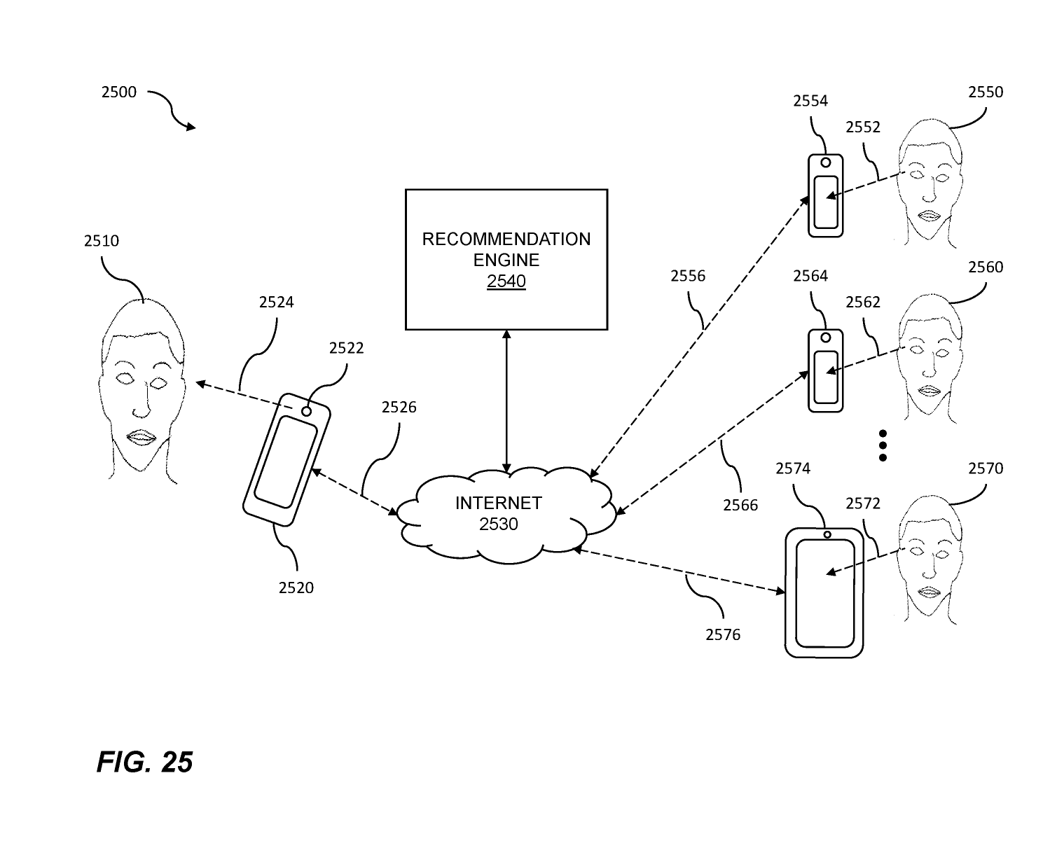

FIG. 25 shows an example live-streaming social video scenario. Live-streaming video is an example of one-to-many social media where video can be sent over the Internet from one person to a plurality of people using a social media app and/or platform. Live-streaming is one of numerous popular techniques used by people who want to disseminate ideas, send information, provide entertainment, share experiences, and so on. Some of the live streams can be scheduled, such as webcasts, online classes, sporting events, news, computer gaming, or video conferences, while others can be impromptu streams that are broadcast as and when needed or desirable. Examples of impromptu live-stream videos can range from individuals simply wanting to share experiences with their social media followers, to coverage of breaking news, emergencies, or natural disasters. This latter coverage is known as mobile journalism or “mo jo” and is becoming increasingly commonplace. “Reporters” can use networked, portable electronic devices to provide mobile journalism content to a plurality of social media followers. Such reporters can be quickly and inexpensively deployed as the need or desire arises.

Several live-streaming social media apps and platforms can be used for transmitting video. One such video social media app is Meerkat™ that can link with a user's Twitter™ account. Meerkat™ enables a user to stream video using a handheld, networked, electronic device coupled to video capabilities. Viewers of the live stream can comment on the stream using tweets that can be seen by and responded to by the broadcaster. Another popular app is Periscope™ that can transmit a live recording from one user to that user's Periscope™ or other social media followers. The Periscope™ app can be executed on a mobile device. The user's followers can receive an alert whenever that user begins a video transmission. Another live-stream video platform is Twitch which can be used for video streaming of video gaming, and broadcasts of various competitions, concerts and other events.

The example 2500 shows user 2510 broadcasting a video live-stream to one or more people 2550, 2560, 2570, and so on. A portable, network-enabled electronic device 2520 can be coupled to a camera 2522 that is forward facing or front facing. The portable electronic device 2520 can be a smartphone, a PDA, a tablet, a laptop computer, and so on. The camera 2522 coupled to the device 2520 can have a line-of-sight view 2524 to the user 2510 and can capture video of the user 2510. The captured video can be sent to a recommendation engine 2540 using a network link 2526 to the Internet 2530. The network link can be a wireless link, a wired link, and so on. The recommendation engine 2540 can recommend to the user 2510 an app and/or platform that can be supported by the server and can be used to provide a video live-stream to one or more followers of the user 2510. The example 2500 shows three followers of the user 2510, followers 2550, 2560, and 2570. Each follower has a line-of-sight view to a video screen on a portable, networked electronic device. In other embodiments, one or more followers follow the user 2510 using any other networked electronic device, including a computer. In the example 2500, the person 2550 has a line-of-sight view 2552 to the video screen of a device 2554, the person 2560 has a line-of-sight view 2562 to the video screen of a device 2564, and the person 2570 has a line-of-sight view 2572 to the video screen of a device 2574. The portable electronic devices 2554, 2564, and 2574 each can be a smartphone, a PDA, a tablet, and so on. Each portable device can receive the video stream being broadcast by the user 2510 through the Internet 2530 using the app and/or platform that can be recommended by the recommendation engine 2540. The device 2554 can receive a video stream using the network link 2556, the device 2564 can receive a video stream using the network link 2566, the device 2574 can receive a video stream using the network link 2576, and so on. The network link can be a wireless link, and wired link, and so on. Depending on the app and/or platform that can be recommended by the recommendation engine 2540, one or more followers, such as the followers 2550, 2560, 2570, and so on, can reply to, comment on, and otherwise provide feedback to the user 2510 using their devices 2554, 2564, and 2574 respectively.

As described above, one or more videos of various types, including live-streamed videos, can be presented to a plurality of users for wide ranging purposes. These purposes can include, but are not limited to, entertainment, education, general information, political campaign messages, social media sharing, and so on. Mental state data can be collected from the one or more users as they view the videos. The collection of the mental state data can be based on a user agreeing to enable a camera that can be used for the collection of the mental state data. The collected mental state data can be analyzed for various purposes. When the mental state data has been collected from a sufficient number of users to enable anonymity, then the aggregated mental state data can be used to provide information on aggregated mental states of the viewers. The aggregated mental states can be used to recommend videos that can include media presentations, for example. The recommendations of videos can be based on videos that can be similar to those videos to which a user had a particular mental state response, for example. The recommendations of videos can include videos to which the user can be more likely to have a favorable mental state response, videos that can be enjoyed by the user's social media contacts, videos that can be trending, and so on.

The aggregated mental state data can be represented using a variety of techniques and can be presented to the one or more users. The aggregated mental state data can be presented while the one or more users are viewing the video, and the aggregated mental state data can be presented after the one or more users have viewed the video. The video can be obtained from a server, a collection of videos, a live-stream video, and so on. The aggregated mental state data can be presented to the users using a variety of techniques. For example, the aggregated mental state data can be displayed as colored dots, as graphs, etc. The colored dots, graphs, and so on, can be displayed with the video, embedded in the video, viewed subsequently to viewing the video, or presented in another fashion. The aggregated mental state data can also be used to provide feedback to the originator of the video, where the feedback can include viewer reaction or reactions to the video, receptiveness to the video, effectiveness of the video, etc. The aggregated mental state data can include sadness, happiness, frustration, confusion, disappointment, hesitation, cognitive overload, focusing, being engaged, attending, boredom, exploration, confidence, trust, delight, valence, skepticism, satisfaction, and so on. The videos can include live-streamed videos. The videos and the live-streamed videos can be presented along with the aggregated mental state data from the one or more users. The aggregated mental state data, as viewed by the users, can be employed by the same users to determine what mental states are being experienced by other users as all parties view a given video, when those mental states occur, whether those mental states are similar to the one or more mental states experienced by the users, and so on. The viewing of the aggregated mental state data can enable a viewer to experience videos viewed by others, to feel connected to other users who are viewing the videos, to share in the experience of viewing the videos, to gauge the mental states experienced by the users, and so on.

The collecting of mental state data can be performed as one or more users observe the videos described above. For example, a news site, a social media site, a crowdsourced site, an individual's digital electronic device, and so on can provide the videos. The mental state data can be collected as the one or more users view a given video or live-stream video. The mental state data can be recorded and analyzed. The results of the analysis of the collected mental state data from the one or more users can be displayed to the one or more users following the viewing of the video, for example. For confidentiality reasons, mental state data can be collected from a minimum or threshold number of users before the aggregated mental state data is displayed. One or more users on one or more social media sites can share their individual mental state data and the aggregated mental state data that can be collected. For example, a user could share with their Facebook™ friends her or his mental state data results from viewing a particular video. How a user responds to a video can be compared to the responses of their friends, of other users, and so on, using a variety of techniques including a social graph. For example, the user could track the reactions of her or his friends to a particular video using a Facebook™ social graph. The mental state data can be shared automatically or can be shared manually, as selected by the user. Automatic sharing of mental state data can be based on user credentials such as logging in to a social media site. A user's privacy can also be enabled using a variety of techniques, including anonymizing a user's mental state data, anonymizing and/or deleting a user's facial data, and so on. Facial tracking data can be provided in real time. In embodiments, the user has full control of playback of a video, a streamed video, a live-streamed video, and so on. That is, the user can pause, skip, scrub, go back, stop, and so on. Recommendations can be made to the user regarding viewing another video. The flow of a user viewing a video can continue from the current video to another video based on the recommendations. The next video can be a streamed video, a live-streamed video, and so on.

In another embodiment, aggregated mental state data can be used to assist a user to select a video, video stream, live-stream video, and so on, that can be considered most engaging to the user. By way of example, if there is a user who is interested in a particular type of video stream such as a gaming stream, a sports stream, a news stream, a movie stream, and so on, and that favorite video stream is not currently available to the user, then recommendations can be made to the user based on a variety of criteria to assist in finding an engaging video stream. For example, the user can connect to a video stream that is presenting one or more sports events, but if the stream does not include the stream of the user's favorite, then recommendations can be made to the user based on aggregated mental state data of other users who are ranking or reacting to the one or more sports events currently available. Similarly, if analysis of the mental state data collected from the user indicates that the user is not reacting favorably to a given video stream, then a recommendation can be made for another video stream based on an audience who is engaged with the latter stream.

A given user can choose to participate in collection of mental state data for a variety of purposes. One or more personae can be used to characterize or classify a given user who views one or more videos. The personae can be useful for recommending one or more videos to a user based on mental state data collected from the user, for example. The recommending of one or more videos to the user can be based on aggregated mental state data collected from one or more users with a similar persona. Many personae can be described and chosen based on a variety of criteria. For example, personae can include a demo user, a social sharer, a video viewing enthusiast, a viral video enthusiast, an analytics research, a quantified self-user, a music aficionado, and so on. Any number of personae can be described, and any number of personae can be assigned to a particular user.

A demo user can be a user who is curious about the collection of mental state data and the presentation of that mental state data. The demo user can view any number of videos in order to experience the mental state data collection and to observe their own social curve, for example. The demo user can view some viral videos in order to observe an aggregated population. The demo user can be interested in trying mental state data collection and presentation in order to determine how she or he would use such a technique for their own purposes.

A social sharer can be a user who is enthusiastic about sharing demos and videos with their friends. The friends can be social media friends such as Facebook™ friends, for example. The videos can be particularly engaging, flashy, slickly produced, and so on. The social sharer can be interested in the reactions to and the sharing of the video that the social sharer has shared. The social sharer can also compare their own mental states to those of their friends. The social sharer can use the comparison to increase their knowledge of their friends and to gather information about the videos that those friends enjoyed.

A video-viewing enthusiast can be a user who enjoys watching videos and desires to watch more videos. Such a persona can generally stay within the context of a video streaming site, for example. The viewing by the user can be influenced by recommendations that can draw the user back to view more videos. When the user finds that the recommendations are desirable, then the user will likely continue watching videos within the streaming site. The video enthusiast can want to find the videos that the user wants to watch and also the portions of the videos that the user wants to watch.

A viral video enthusiast can be a user who chooses to watch many videos through social media. The social media can include links, shares, comments, etc. from friends of the user, for example. When the user clicks on the link to the video, the user can be connected from the external site to the video site. For example, the user can click a link in Reddit™ Twitter™, Facebook™, etc. and be connected to a video on YouTube™ or another video sharing site. Such a user is interested in seamless integration between the link on the social media site and the playing of the video on the video streaming site. The video streaming site can be a live-streaming video site.

An analytics researcher or “uploader” can be a user who can be interested in tracking video performance of one or more videos over time. The performance of the one or more videos can be based on various metrics, including emotional engagement of one or more viewers as they view the one or more videos. The analytics researcher can be interested primarily in the various metrics that can be generated based on a given video. The analytics can be based on demographic data, geographic data, and so on. Analytics can also be based on trending search terms, popular search terms, and so on, where the search terms can be identified using web facilities such as Google Trends™.

A quantified self-user can be a user who can be interested in studying and/or documenting her or his own video watching experiences. The qualified self-user reviews her or his mental state data over time, can sort a list of viewed videos over a time period, and so on. The qualified self-user can compare their mental state data that is collected while watching a given video with their personal norms. This user persona can also provide feedback. The quantified self-user can track their reactions to one or more videos over time and over videos, where tracking over videos can include tracking favorite videos, categorizing videos that have been viewed, remembering favorite videos, etc.

A music enthusiast can be a user who is a consumer of music and who uses a video streaming site such as a music streaming site. For example, this user persona can use music mixes from sites such as YouTube™ as if they were provided by a music streaming site such as Spotify™, Pandora™, Apple Music™, Tidal™, and so on. The music enthusiast persona can be less likely to be sitting in front of a screen, since their main mode of engagement is sound rather than sight. Facial reactions that can be captured from the listener can be weaker, for example, than those facial reactions captured from a viewer.

The method can include comparing the mental state data that was captured against mental state event temporal signatures. In embodiments, the method includes identifying a mental state event type based on the comparing. The recommending of the second media presentation can be based on the mental state event type. The recommending of the second media presentation can be performed using one or more processors. The first media presentation can include a first socially shared live-stream video. The method can further comprise generating highlights for the first socially shared live-stream video, based on the mental state data that was captured. The first socially shared live-stream video can include an overlay with information on the mental state data that was captured. The overlay can include information on the mental state data collected from the other people. The mental state data that was captured for the first socially shared live-stream video can be analyzed substantially in real time. In some embodiments, the second media presentation includes a second socially shared live-stream video. The method can further comprise a recommendation for changing from the first socially shared live-stream video to the second socially shared live-stream video. The first socially shared live-stream video can be broadcast to a plurality of people. In embodiments, the method further comprises providing an indication to the individual that the second socially shared live-stream video is ready to be joined.

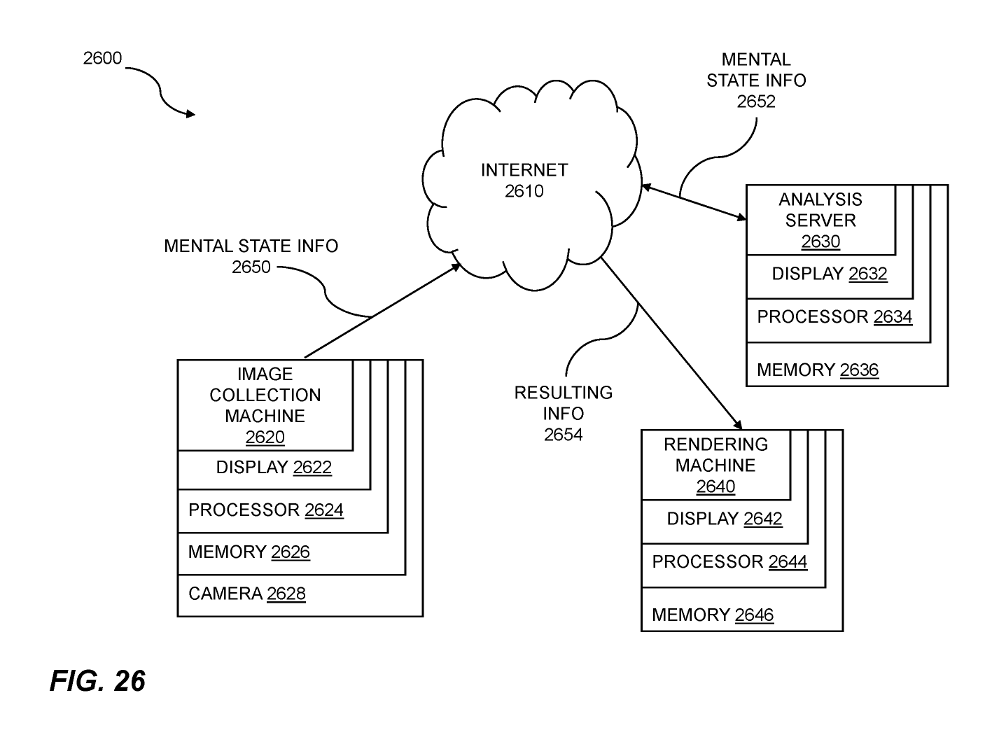

FIG. 26 is a system diagram for analyzing mental state information. The mental state information analysis can include analyzing emotional content from a plurality of images for a plurality of participants. The system 2600 can be implemented using one or more machines. The system 2600 includes aspects of image collection, image analysis, and rendering. The system 2600 can include a memory which stores instructions and one or more processors attached to the memory wherein the one or more processors, when executing the instructions which are stored, are configured to: capture data on an individual into a computer system, wherein the data provides information for evaluating a mental state of the individual, and wherein the data includes facial data for the individual; perform image analysis on the facial data, wherein the image analysis includes inferring mental states; compare the data that was captured for the individual against a plurality of mental state event temporal signatures; receive analysis from a web server, wherein the analysis is based on the data on the individual which was captured and the comparing the data that was captured for the individual against a plurality of mental state event temporal signatures; and render an output which describes the mental state of the individual based on the analysis which was received. The system 2600 can perform a computer-implemented method for analysis comprising: capturing data on an individual into a computer system, wherein the data provides information for evaluating a mental state of the individual, and wherein the data includes facial data for the individual; perform image analysis on the facial data, wherein the image analysis includes inferring mental states; comparing the data that was captured for the individual against a plurality of mental state event temporal signatures; receiving analysis from a web server, wherein the analysis is based on the data on the individual which was captured and the comparing the data that was captured for the individual against a plurality of mental state event temporal signatures; and rendering an output which describes the mental state of the individual based on the analysis which was received.

The system 2600 can include one or more image data collection machines 2620 linked to an analysis server 2630 and a rendering machine 2640 via the Internet 2610 or another computer network. The network can be wired or wireless, a combination of wired and wireless networks, and so on. Mental state information 2652 can be transferred to the analysis server 2630 through the Internet 2610, for example. The example image data collection machine 2620 shown comprises one or more processors 2624 coupled to a memory 2626 which can store and retrieve instructions, a display 2622, and a camera 2628. The camera 2628 can include a webcam, a video camera, a still camera, a thermal imager, a CCD device, a phone camera, a three-dimensional camera, a depth camera, a light field camera, a plenoptic camera, multiple webcams used to show different views of a person, or any other type of image capture technique that can allow captured data to be used in an electronic system. The memory 2626 can be used for storing instructions, image data on a plurality of people, gaming data, one or more classifiers, one or more actions units, and so on. The display 2622 can be any electronic display, including but not limited to, a computer display, a laptop screen, a net-book screen, a tablet computer screen, a smartphone display, a mobile device display, a remote with a display, a television, a projector, or the like. Mental state data 2650 can be transferred via the Internet 2610 for a variety of purposes including analysis, rendering, storage, cloud storage, sharing, social sharing, and so on.

The analysis server 2630 can include one or more processors 2634 coupled to a memory 2636 which can store and retrieve instructions, and it can also include a display 2632. The analysis server 2630 can receive the analytics for live streaming and mental state information 2652 and can analyze the image data using classifiers, action units, and so on. The classifiers and action units can be stored in the analysis server, loaded into the analysis server, provided by a user of the analysis server, and so on. The analysis server 2630 can use image data received from the image data collection machine 2620 to produce resulting information 2654. The resulting information can include an emotion, a mood, a mental state, etc., and can be based on the analytics for live streaming. In some embodiments, the analysis server 2630 receives image data from a plurality of image data collection machines, aggregates the image data, processes the image data or the aggregated image data, and so on.

The rendering machine 2640 can include one or more processors 2644 coupled to a memory 2646 which can store and retrieve instructions and data, and it can also include a display 2642. The rendering of the resulting information rendering data 2654 can occur on the rendering machine 2640 or on a different platform from the rendering machine 2640. In embodiments, the rendering of the resulting information rendering data 2654 occurs on the image data collection machine 2620 or on the analysis server 2630. As shown in the system 2600, the rendering machine 2640 can receive resulting information rendering data 2654 via the Internet 2610 or another network from the image data collection machine 2620, from the analysis server 2630, or from both. The rendering can include a visual display or any other appropriate display format.

The system 2600 can include a computer program product stored on a non-transitory computer-readable medium for analyzing mental states, the computer program product comprising code which causes one or more processors to perform operations of: capturing data on an individual into a computer system, wherein the data provides information for evaluating a mental state of the individual, and wherein the data includes facial data for the individual; performing image analysis on the facial data, wherein the image analysis includes inferring mental states; comparing the data that was captured for the individual against a plurality of mental state event temporal signatures; receiving analysis from a web server, wherein the analysis is based on the data on the individual which was captured and the comparing the data that was captured for the individual against a plurality of mental state event temporal signatures; and rendering an output which describes the mental state of the individual based on the analysis which was received.

The above methods may be executed on one or more processors on one or more computer systems. Embodiments may include various forms of distributed computing, client/server computing, and cloud based computing. Further, it will be understood that for the flow diagrams in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flow diagram illustrations depict methods, apparatus, systems, and computer program products. Each element of the block diagrams and flow diagram illustrations, as well as each respective combination of elements in the block diagrams and flow diagram illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, by a computer system, and so on. Any and all of which may be generally referred to herein as a “circuit,” “module,” or “system.”

A programmable apparatus which executes any of the above mentioned computer program products or computer implemented methods may include one or more processors, microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that can include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM), Flash, MRAM, FeRAM, phase change memory, an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

It will be appreciated that computer program instructions may include computer executable code. A variety of languages for expressing computer program instructions can include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads can be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the entity causing the step to be performed.

While the invention has been disclosed in connection with preferred embodiments shown and described in detail, various modifications and improvements thereon will become apparent to those skilled in the art. Accordingly, the spirit and scope of the present invention is not to be limited by the foregoing examples, but is to be understood in the broadest sense allowable by law.

What is claimed is:

1. A computer implemented method for analyzing mental states comprising:
capturing data on an individual into a computer system, wherein the data provides information for evaluating a mental state of the individual, and wherein the data includes facial data for the individual;
performing image analysis on the facial data, wherein the image analysis includes inferring mental states;
comparing the data that was captured for the individual against a plurality of mental state event temporal signatures;
receiving analysis from a web server, wherein the analysis is based on the data on the individual which was captured and the comparing the data that was captured for the individual against a plurality of mental state event temporal signatures; and
rendering an output which describes the mental state of the individual based on the analysis which was received.

2. The method of claim 1 further comprising matching a first event signature, from the plurality of mental state event temporal signatures, against the data that was captured wherein the rendering of the output is based on the matching of the first event signature.

3. The method of claim 1 further comprising performing unsupervised clustering of features extracted from the facial data.

4. The method of claim 1 further comprising analyzing the data to produce mental state information.

5. The method of claim 4 wherein the analyzing the data is further based on a demographic basis.

6. The method of claim 1 further comprising identifying a first face and a second face within the facial data.

7. The method of claim 6 further comprising determining weights and image classifiers, wherein the determining is performed on a remote server based on the facial data including the first face and the second face.

8. The method of claim 1 wherein the data on the individual includes facial expressions, physiological information, or accelerometer readings.

9-10. (canceled)

11. The method of claim 8 wherein the physiological information is collected without contacting the individual.

12. The method of claim 1 wherein the mental state is one of a cognitive state and an emotional state.

13. The method of claim 1 wherein the facial data includes information on facial expressions, action units, head gestures, smiles, squints, lowered eyebrows, raised eyebrows, smirks, or attention.

14. The method of claim 1 further comprising inferring mental states, based on the data which was collected and the analysis of the facial data.

15-16. (canceled)

17. The method of claim 1 further comprising indexing the data on the individual through the web server.

18. (canceled)

19. The method of claim 1 further comprising receiving analysis information on a plurality of other people wherein the analysis information allows evaluation of a collective mental state of the plurality of other people.

20. The method of claim 19 wherein the analysis information includes correlation for the mental state of the plurality of other people to the data which was captured on the mental state of the individual.

21. The method of claim 20 wherein the correlation is based on metadata from the individual and metadata from the plurality of other people.

22. The method of claim 20 wherein the correlation is based on the comparing the data that was captured for the individual against a plurality of mental state event temporal signatures.

23. The method of claim 1 wherein the analysis which is received from the web server is based on specific access rights.

24. The method of claim 1 further comprising sending a request to the web server for the analysis.

25. (canceled)

26. The method of claim 1 further comprising sending a subset of the data which was captured on the individual to the web server.

27. The method of claim 1 wherein the rendering is based on data which is received from the web server.

28. The method of claim 27 wherein the data which is received includes a serialized object in a form of JavaScript Object Notation (JSON).

29. The method of claim 28 further comprising de-serializing the serialized object into a form for a JavaScript object.

30. The method of claim 1 wherein the rendering further comprises recommending a course of action based on the mental state of the individual.

31. (canceled)

32. A computer program product stored on a non-transitory computer-readable medium for analyzing mental states, the computer program product comprising code which causes one or more processors to perform operations of:
capturing data on an individual into a computer system, wherein the data provides information for evaluating a mental state of the individual, and wherein the data includes facial data for the individual;
performing image analysis on the facial data, wherein the image analysis includes inferring mental states;
comparing the data that was captured for the individual against a plurality of mental state event temporal signatures;
receiving analysis from a web server, wherein the analysis is based on the data on the individual which was captured and the comparing the data that was captured for the individual against a plurality of mental state event temporal signatures; and
rendering an output which describes the mental state of the individual based on the analysis which was received.

33. A system for analyzing mental states comprising:
a memory which stores instructions;
one or more processors attached to the memory wherein the one or more processors, when executing the instructions which are stored, are configured to:
capture data on an individual into a computer system, wherein the data provides information for evaluating a mental state of the individual, and wherein the data includes facial data for the individual;
perform image analysis on the facial data, wherein the image analysis includes inferring mental states;
compare the data that was captured for the individual against a plurality of mental state event temporal signatures;
receive analysis from a web server, wherein the analysis is based on the data on the individual which was captured and compared data that was captured for the individual against a plurality of mental state event temporal signatures; and
render an output which describes the mental state of the individual based on the analysis which was received.
